(19) United States
(12) Patent Application Publication
(10) Pub. No.: US 2015/0167105 A1
(43) Pub. Date: Jun. 18, 2015

(54) GENETIC LOCI ASSOCIATED WITH GRAY LEAF SPOT IN MAIZE

(71) Applicant: DOW AGROSCIENCES LLC, INDIANAPOLIS, IN (US)

(72) Inventors: Jafar Mammadov, Carmel, IN (US); Jerry R. Rice, Evansville, IN (US); Wei Chen, Carmel, IN (US); Yanxin Star Gao, Waunakee, WI (US); Joseph T. Metzler, Homer, IL (US); Ruihua Ren, Carmel, IN (US)

(21) Appl. No.: 14/561,496

(22) Filed: Dec. 5, 2014

Related U.S. Application Data
(60) Provisional application No. 61/916,970, filed on Dec. 17, 2013.

(51) Int. Cl. C12Q 1/68; A01H 5/10; A01H 1/04

(52) U.S. Cl. CPC C12Q 1/6895; A01H 1/04; A01H 5/10; C12Q 2600/16; C12Q 2600/13; C12Q 2600/156

(57) ABSTRACT

This invention relates to methods for identifying maize plants that have decreased gray leaf spot. The methods use molecular markers to identify and to select plants with decreased gray leaf spot or to identify and deselect plants with increased gray leaf spot. Maize plants generated by the methods of the invention are also a feature of the invention.

### CROSS-REFERENCE TO RELATED APPLICATION

This application claims the benefit of U.S. Provisional Application No. 61/916,970, which was filed in the U.S. Patent and Trademark Office on Dec. 17, 2013, the entirety of the disclosure of which is expressly incorporated by reference herein.

### FIELD OF THE INVENTION

The present invention relates to methods useful in decreasing Gray Leaf Spot in maize plants.

### BACKGROUND OF THE INVENTION

Gray Leaf Spot [GLS, causal agent Cercospora zeae-maydis (Tehon and Daniels 1925)] is one of the most important foliar diseases of maize in all areas where the crop is being cultivated. The severity of GLS depends on climate conditions suitable for fungus development. The disease is prevalent in the areas where dewy mornings are followed by hot humid afternoons and relatively cool nights. In the USA, the damage to maize from GLS had been mild up to the 1970s. However, the introduction of reduced tillage practice as a measure to fight soil erosion created favorable conditions for the pathogen to over winter in the corn field and re-infect plants in the summer (Ward et al. 1999). As it was predicted in the early 1980s, during last 20 years the importance of GLS in the USA has increased (Latterell and Rossi 1983). Although in the USA the situation with GLS severity is not as critical as in sub-Saharan Africa or Brazil, evidence of climate change, increasing corn monoculture as well as narrow North American resistant germplasm can turn the disease into a serious threat to US corn production. In order to control the disease, the development of GLS-resistant corn varieties can ensure the security of corn production in the USA.

In late 1980s, the first studies pertaining to the inheritance of GLS resistance were reported in the scientific literature. The studies showed that the resistance to the disease was highly heritable and conditioned mainly by additive effects (Donahue et al. 1991; Thompson et al. 1987; Ulrich et al. 1990). In the beginning of the 1990s, it became obvious that in maize the resistance to GLS was controlled by quantitative trait loci (QTL) (Bubeck et al. 1993; Maroof et al. 1996). During the last 20 years, using various sources of resistance, types of mapping populations, molecular markers and environments, over 57 QTL were detected in all 10 chromosomes of maize. Using the meta-analysis approach, Shi et al. (2007) hypothesized that only 26 out of 57 were true QTL with seven consensus QTL across all studies. According to Shi et al (2007) the consensus QTL were located in chromosome bins 1.06, 2.06, 3.04, 4.06, 4.08, 5.03, and 8.06. Further reports also confirmed that GLS resistance was highly heritable (Coates and White 1998; Gevers et al. 1994; Gordon et al. 2006).

However, despite the substantial number of GLS QTL mapping efforts, the majority of them have had one major limitation, which is the low resolution of bi-parental mapping populations. In recent GLS QTL mapping studies, the sizes of bi-parental mapping populations ranged between 100-300 individuals (Balint-Kurti et al. 2008; Zwonitzer et al. 2010). Although the bi-parental genetic mapping approach offers high QTL detection power, its resolution remains low due to inaccurate recombination information (Bennewitz et al. 2002). This problem leads to a strong statistical association of QTL with blocks of markers that physically span large chromosomal segments. To capture all possible recombination events, one can increase the sizes of mapping populations, which is a very time- and cost-intensive procedure especially if it is dealt with immortal populations such as recombinant inbred lines (RILs) or double haploids (DH). However, even fine mapping in many cases will not help to delimit QTL intervals to fairly smaller segments of DNA because of limited numbers of meiotic recombinations (Myles et al. 2009). Another way to increase the resolution within a QTL confidence interval and discover additional recombination events was proposed to be the application of high-density marker technologies, e.g. polymorphisms derived from genotyping-by-sequencing (GBS) (Pan et al. 2012). According to Pan et al. (2012), in his research work GBS markers facilitated the discovery of additional recombination breakpoints.

In contrast to the bi-parental approach, a linkage disequilibrium-based genome-wide association study (GWAS) overcomes the problem related to the lack of recombination events due to the structure of the association mapping population, which is composed of genetically un-related individuals with unknown pedigrees and accumulates a larger number of historical recombination events that occurred in the past (Nordborg and Tavaré 2002). However, unlike the bi-parental approach of QTL mapping, the detection power of GWAS is fairly low and the method is prone to discover false-positive QTL (Aranzana et al. 2005). The high rate of false-positive QTL detection, however, could be conditioned by the limitation of current GWAS analysis as it is based on the single-marker analysis. Single-marker analysis has several disadvantages including 1) limitation of discovering the polygenic feature of complex traits, 2) the incapability of exploring gene interactions, and 3) inability of revealing the underlying genetic architecture of the complex traits.

Despite the fact that information for GLS resistance QTL is available in the art and resistant and tolerant genotypes have been reported, few can be classified as highly resistant and there is little evidence of any strong resistance to GLS in commercially available hybrids. There need remains for commercially acceptable hybrids that are GLS resistant and for a method to develop and track resistant maize inbreds and hybrids through marker assisted breeding.

Described within is a method to map GLS resistance QTL using GWAS approach. The GWAS approach used in this study was based on a proprietary model that was designed internally at DAS to overcome all the above-mentioned disadvantages that are the characteristic of existing GWAS models, particularly single-marker analysis.

The present invention allows the selection of progeny, which contains the genomic background of the agronomically desirable parent and the genomic trait of the GLS resistant donor parent. The present invention also allows tracking the GLS resistance QTL in order to introgress the GLS resistance trait into new plants through traditional marker-assisted breeding.

### SUMMARY OF THE INVENTION

In one embodiment, methods of identifying a maize plant that displays increased GLS resistance, comprising detecting in germplasm of the maize plant at least one allele of a marker locus are provided. The marker locus can be selected from two marker loci found on chromosome 1 and are located within two chromosomal intervals (1.1-1.2) comprising and flanked by (1.1) PZE-101025686 and PZE-101026265; (1.2) DAS-PZ-14748 and bz2-2; and at least one allele within each chromosomal interval is associated with increased GLS resistance. The two marker loci can be (1.1) chr1_{—}15269379; and (1.2) PZE-101188909, as well as any other marker that is linked to these markers. Maize plants identified by this method are also of interest.

In another embodiment, methods of identifying a maize plant that displays increased GLS resistance, comprising detecting in germplasm of the maize plant at least one allele of a marker locus are provided. The marker locus can be selected from six marker loci found on chromosome 2 and are located within six chromosomal intervals (2.1-2.6) comprising and flanked by (2.1) PZE-102013511 and DAS-PZ-32659; (2.2) PZE-102040682 and Mo17-12859; (2.3) PZE-102070420 and Mo17-13313; (2.4) PZE-102072947 and PZE-102073407; (2.5) PZE-102078235 and PZE-102079631; (2.6) PZE-102088257 and PZE-102103382; and at least one allele within each chromosomal interval is associated with decreased GLS. The six marker loci can be (2.1) chr2_{—}6858691; (2.2) PZE-102041193; (2.3) PZE-102072013; (2.4) chr2_{—}44697986; (2.5) chr2_{—}44697986; and (2.6) PZE-102088902, as well as any other marker that is linked to these markers. Maize plants identified by this method are also of interest.

In another embodiment, methods of identifying a maize plant that displays increased GLS resistance, comprising detecting in germplasm of the maize plant at least one allele of a marker locus are provided. The marker locus is located within a chromosomal interval comprising and flanked by PZE-103052576 and PZE-103057593; and at least one allele is associated with decreased GLS. The marker locus can be PZE-103053562, as well as any other marker that is linked to this marker. The marker locus can be found on chromosome 3, within the interval comprising and flanked by PZE-103052576 and PZE-103057593, and comprises at least one allele that is associated with decreased GLS. Maize plants identified by this method are also of interest.

In another embodiment, methods of identifying a maize plant that displays increased GLS resistance, comprising detecting in germplasm of the maize plant at least one allele of a marker locus are provided. The marker locus can be selected from two marker loci found on chromosome 4 and are located within two chromosomal intervals (4.1-4.2) comprising and flanked by (4.1) PZE-104093278 and DAS-PZ-8846 and (4.2) DSDS0099-1 and PZE-104105141, and at least one allele within each chromosomal interval is associated with decreased GLS. The two marker loci can be (4.1) PZE-104093278 and (4.2) Chr4_{—}180264145, as well as any other marker that is linked to these markers. Maize plants identified by this method are also of interest.

In another embodiment, methods of identifying a maize plant that displays increased GLS resistance, comprising detecting in germplasm of the maize plant at least one allele of a marker locus are provided. The marker locus can be selected from two marker loci found on chromosome 5 and located within the interval comprising and flanked by PZE-105166071 and DAS-PZ-14276, and comprises at least one allele that is associated with increased GLS resistance. The marker locus can be PZE-105165816, as well as any other marker that is linked to this marker. Maize plants identified by this method are also of interest.

In another embodiment, methods of identifying a maize plant that displays increased GLS resistance, comprising detecting in germplasm of the maize plant at least one allele of a marker locus are provided. The marker locus can be selected from two marker loci found on chromosome 6, which are located within two chromosomal intervals (6.1-6.2) comprising and flanked by (6.1) DAS-PZ-18055 and PZE-106101510 and (6.2) Mo17-12530 and Mo17-14401, and at least one allele within each chromosomal interval is associated with increased GLS resistance. The two marker loci can be (6.1) PZE-106100504 and (6.2) PZE-106107639, as well as any other marker that is linked to these markers. Maize plants identified by this method are also of interest.

In another embodiment, methods of identifying a maize plant that displays increased GLS resistance, comprising detecting in germplasm of the maize plant at least one allele of a marker locus are provided. The marker locus can be selected from two marker loci found on chromosome 7, which are located within two chromosomal intervals (7.1-7.2) comprising and flanked by (7.1) PZE-107004762 and PZE-107004893 and (7.2) DAS-PZ-11250 and PHM4080.15, and at least one allele within each chromosomal interval is associated with increased GLS resistance. The two marker loci can be (7.1) PZE-107004786 and (7.2) PZE-107020739, as well as any other marker that is linked to these markers. Maize plants identified by this method are also of interest.

In another embodiment, methods of identifying a maize plant that displays increased GLS resistance, comprising detecting in germplasm of the maize plant at least one allele of a marker locus are provided. The marker locus can be selected from three marker loci found on chromosome 8, which are located within three chromosomal intervals (8.1-8.3) comprising and flanked by (8.1) PZE-108006063 and PZE-108006412; (8.2) PZE-108020151 and PZE-108020416; (8.3) PZE-108022528 and PZE-108023337; and at least one allele within each chromosomal interval is associated with decreased GLS. The three marker loci can be (8.1) chr8_{—}7675588; (8.2) PZE-108020413; and (8.3) PZE-108022834, as well as any other marker that is linked to these markers. Maize plants identified by this method are also of interest.

In another embodiment, methods of identifying a maize plant that displays increased GLS resistance, comprising detecting in germplasm of the maize plant at least one allele of a marker locus are provided. The marker locus can be selected from nine marker loci found on chromosome 8 and are located within one chromosomal interval comprising and flanked by PZE-108047170 and PZE-108051324; and at least one allele of each marker loci within the chromosomal interval is associated with increased GLS resistance. The nine marker loci can be (8.4) PZE-108047366; (8.5) GLS_chr8_{—}80296742; (8.6) GLS_chr8_{—}80499765; (8.7) PZE-108048175; (8.8) PZE-108048978; (8.9) GLS_chr8_{—}83335579; (8.10) GLS_chr8_{—}86463733; (8.11) GLS_chr8_{—}87640198; and (8.12) PZE-108050255, as well as any other marker that is linked to these markers. Maize plants identified by this method are also of interest.

In another embodiment, methods of identifying a maize plant that displays increased GLS resistance, comprising detecting in germplasm of the maize plant at least one allele of a marker locus are provided. The marker locus can be selected from two marker loci found on chromosome 9, which are located within two chromosomal intervals (9.1-9.2) comprising and flanked by (9.1) PZE-109016836 and PZE-109017324 and (9.2) PZE-109083580 and PZE-109084648, and at least one allele within each chromosomal interval is associated with decreased GLS. The two marker loci can be (9.1) PZE-109017122 and (9.2) PZE-109084575, as well as any other marker that is linked to these markers. Maize plants identified by this method are also of interest.

In another embodiment, methods of identifying a maize plant that displays increased GLS resistance, comprising detecting in germplasm of the maize plant at least one allele of a marker locus are provided. The marker locus can be selected from two marker loci found on chromosome 10, which are located within two chromosomal intervals (10.1-10.2) comprising and flanked by (10.1) PZE-110000036 and PZE-110000803 and (10.2) PZE-110000803 and PZE-110001270, and at least one allele within each chromosomal interval is associated with increased GLS resistance. The two marker loci can be (10.1) PZE-110000028 and (10.2) PZE-110000899, as well as any other marker that is linked to these markers. Maize plants identified by this method are also of interest.

In another embodiment, methods for identifying maize plants with increased GLS resistance by detecting a haplotype in the germplasm of the maize plant are provided. The haplotype comprises alleles at one or more marker loci, wherein the one or more marker loci are found on chromosome 1 and are selected from the group consisting of chr1_{—}15269379 and PZE-101188909. The haplotype is associated with increased GLS resistance.

In another embodiment, methods for identifying maize plants with increased GLS resistance by detecting a haplotype in the germplasm of the maize plant are provided. The haplotype comprises alleles at one or more marker loci, wherein the one or more marker loci are found on chromosome 2 and are selected from the group consisting of chr2_{—}6858691, PZE-102041193, PZE-102072013, chr2_{—}44697986, PZE-102079279, and PZE-102088902. The haplotype is associated with decreased GLS.

In another embodiment, methods for identifying maize plants with decreased GLS susceptibility by detecting a haplotype in the germplasm of the maize plant are provided. The haplotype comprises alleles at one or more marker loci, wherein the one or more marker loci are found on chromosome 3 and are selected from the group consisting of PZE-103053562. The haplotype is associated with decreased GLS.

In another embodiment, methods for identifying maize plants with increased GLS resistance by detecting a haplotype in the germplasm of the maize plant are provided. The haplotype comprises alleles at one or more marker loci, wherein the one or more marker loci are found on chromosome 4 and are selected from the group consisting of PZE-104093278 and Chr4_{—}180264145. The haplotype is associated with decreased GLS.

In another embodiment, methods for identifying maize plants with increased GLS resistance by detecting a haplotype in the germplasm of the maize plant are provided. The haplotype comprises alleles at one or more marker loci, wherein the one or more marker loci are found on chromosome 5 and are selected from the group consisting of PZE-105165816. The haplotype is associated with decreased GLS.

In another embodiment, methods for identifying maize plants with increased GLS resistance by detecting a haplotype in the germplasm of the maize plant are provided. The haplotype comprises alleles at one or more marker loci, wherein the one or more marker loci are found on chromosome 6 and are selected from the group consisting of PZE-106100504 and PZE-106107639. The haplotype is associated with decreased GLS.

In another embodiment, methods for identifying maize plants with increased GLS resistance by detecting a haplotype in the germplasm of the maize plant are provided. The haplotype comprises alleles at one or more marker loci, wherein the one or more marker loci are found on chromosome 7 and are selected from the group consisting of PZE-107004786 and PZE-107020739. The haplotype is associated with decreased GLS.

In another embodiment, methods for identifying maize plants with increased GLS resistance by detecting a haplotype in the germplasm of the maize plant are provided. The haplotype comprises alleles at one or more marker loci, wherein the one or more marker loci are found on chromosome 8 and are selected from the group consisting of chr8_{—}7675588, PZE-108020413, PZE-108022834, PZE-108047366, GLS_chr8_{—}80296742, GLS_chr8_{—}80499765, PZE-108048175, PZE-108048978, GLS_chr8_{—}83335579, GLS_chr8_{—}86463733, GLS_chr8_{—}87640198, and PZE-108050255. The haplotype is associated with decreased GLS.

In another embodiment, methods for identifying maize plants with increased GLS resistance by detecting a haplotype in the germplasm of the maize plant are provided. The haplotype comprises alleles at one or more marker loci, wherein the one or more marker loci are found on chromosome 9 and are selected from the group consisting of PZE-109017122 and PZE-109084575. The haplotype is associated with decreased GLS.

In another embodiment, methods for identifying maize plants with increased GLS resistance by detecting a haplotype in the germplasm of the maize plant are provided. The haplotype comprises alleles at one or more marker loci, wherein the one or more marker loci are found on chromosome 10 and are selected from the group consisting of PZE-110000028 and PZE-110000899. The haplotype is associated with decreased GLS.

In a further embodiment, methods of selecting plants with increased GLS resistance are provided. In one aspect, a first maize plant is obtained that has at least one allele of a marker locus wherein the allele is associated with increased GLS resistance. The marker locus can be selected from two marker loci found on chromosome 1, within two chromosomal intervals (1.1-1.2) comprising and flanked by (1.1) PZE-101025686 and PZE-101026265; (1.2) DAS-PZ-14748 and bz2-2. The first maize plant can be crossed to a second maize plant, and the progeny resulting from the cross can be evaluated for the allele of the first maize plant. Progeny plants that possess the allele from the first maize plant can be selected as having decreased GLS. Maize plants selected by this method are also of interest.

In a further embodiment, methods of selecting plants with increased GLS resistance are provided. In one aspect, a first maize plant is obtained that has at least one allele of a marker locus wherein the allele is associated with decreased GLS. The marker locus can be selected from six marker loci found on chromosome 2, within six chromosomal intervals (2.1-2.6) comprising and flanked by (2.1) PZE-102013511 and DAS-PZ-32659; (2.2) PZE-102040682 and Mo17-12859; (2.3) PZE-102070420 and Mo17-13313; (2.4) PZE-102072947 and PZE-102073407; (2.5) PZE-102078235 and PZE-102079631; (2.6) PZE-102088257 and PZE-102103382. The first maize plant can be crossed to a second maize plant, and the progeny resulting from the cross can be evaluated for the allele of the first maize plant. Progeny plants that possess the allele from the first maize plant can be selected as having decreased GLS. Maize plants selected by this method are also of interest.

In a further embodiment, methods of selecting plants with increased GLS resistance are provided. In one aspect, a first maize plant is obtained that has at least one allele of a marker locus wherein the allele is associated with decreased GLS. The marker locus can be found on chromosome 3, within the interval comprising and flanked by PZE-103052576 and PZE-103057593. The first maize plant can be crossed to a second maize plant, and the progeny resulting from the cross can be evaluated for the allele of the first maize plant. Progeny plants that possess the allele from the first maize plant can be selected as having decreased GLS. Maize plants selected by this method are also of interest.

In a further embodiment, methods of selecting plants with increased GLS resistance are provided. In one aspect, a first maize plant is obtained that has at least one allele of a marker locus wherein the allele is associated with decreased GLS. The marker locus can be selected from two marker loci found on chromosome 4, within two chromosomal intervals (4.1-4.2) comprising and flanked by (4.1) PZE-104093278 and DAS-PZ-8846 and (4.2) DSDS0099-1 and PZE-104105141. The first maize plant can be crossed to a second maize plant, and the progeny resulting from the cross can be evaluated for the allele of the first maize plant. Progeny plants that possess the allele from the first maize plant can be selected as having decreased GLS. Maize plants selected by this method are also of interest.

In a further embodiment, methods of selecting plants with increased GLS resistance are provided. In one aspect, a first maize plant is obtained that has at least one allele of a marker locus wherein the allele is associated with decreased GLS. The marker locus can be found on chromosome 5, within the interval comprising and flanked by PZE-105166071 and DAS-PZ-14276. The first maize plant can be crossed to a second maize plant, and the progeny resulting from the cross can be evaluated for the allele of the first maize plant. Progeny plants that possess the allele from the first maize plant can be selected as having decreased GLS. Maize plants selected by this method are also of interest.

In a further embodiment, methods of selecting plants with increased GLS resistance are provided. In one aspect, a first maize plant is obtained that has at least one allele of a marker locus wherein the allele is associated with decreased GLS. The marker locus can be selected from two marker loci found on chromosome 6, within two chromosomal intervals (6.1-6.2) comprising and flanked by (6.1) DAS-PZ-18055 and PZE-106101510 and (6.2) Mo17-12530 and Mo17-14401. The first maize plant can be crossed to a second maize plant, and the progeny resulting from the cross can be evaluated for the allele of the first maize plant. Progeny plants that possess the allele from the first maize plant can be selected as having decreased GLS. Maize plants selected by this method are also of interest.

In a further embodiment, methods of selecting plants with increased GLS resistance are provided. In one aspect, a first maize plant is obtained that has at least one allele of a marker locus wherein the allele is associated with decreased GLS. The marker locus can be selected from two marker loci found on chromosome 7, within two chromosomal intervals (7.1-7.2) comprising and flanked by (7.1) PZE-107004762 and PZE-107004893 and (7.2) DAS-PZ-11250 and PHM4080.15. The first maize plant can be crossed to a second maize plant, and the progeny resulting from the cross can be evaluated for the allele of the first maize plant. Progeny plants that possess the allele from the first maize plant can be selected as having decreased GLS. Maize plants selected by this method are also of interest.

In a further embodiment, methods of selecting plants with increased GLS resistance are provided. In one aspect, a first maize plant is obtained that has at least one allele of a marker locus wherein the allele is associated with decreased GLS. The marker locus can be selected from three marker loci found on chromosome 8, within three chromosomal intervals (8.1-8.3) comprising and flanked by (8.1) PZE-108006063 and PZE-108006412; (8.2) PZE-108020151 and PZE-108020416; (8.3) PZE-108022528 and PZE-108023337. The first maize plant can be crossed to a second maize plant, and the progeny resulting from the cross can be evaluated for the allele of the first maize plant. Progeny plants that possess the allele from the first maize plant can be selected as having decreased GLS. Maize plants selected by this method are also of interest.

In a further embodiment, methods of selecting plants with increased GLS resistance are provided. In one aspect, a first maize plant is obtained that has at least one allele of a marker locus wherein the allele is associated with decreased GLS. The marker locus can be selected from nine marker loci found on chromosome 8, within one chromosomal interval comprising and flanked by PZE-108047170 and PZE-108051324. The first maize plant can be crossed to a second maize plant, and the progeny resulting from the cross can be evaluated for the allele of the first maize plant. Progeny plants that possess the allele from the first maize plant can be selected as having decreased GLS. Maize plants selected by this method are also of interest.

In a further embodiment, methods of selecting plants with increased GLS resistance are provided. In one aspect, a first maize plant is obtained that has at least one allele of a marker locus wherein the allele is associated with decreased GLS. The marker locus can be selected from two marker loci found on chromosome 9, within two chromosomal intervals (9.1-9.2) comprising and flanked by (9.1) PZE-109016836 and PZE-109017324 and (9.2) PZE-109083580 and PZE-109084648. The first maize plant can be crossed to a second maize plant, and the progeny resulting from the cross can be evaluated for the allele of the first maize plant. Progeny plants that possess the allele from the first maize plant can be selected as having decreased GLS. Maize plants selected by this method are also of interest.

In a further embodiment, methods of selecting plants with increased GLS resistance are provided. In one aspect, a first maize plant is obtained that has at least one allele of a marker locus wherein the allele is associated with decreased GLS. The marker locus can be selected from two marker loci found on chromosome 10, within two chromosomal intervals (10.1-10.2) comprising and flanked by (10.1) PZE-110000036 and PZE-110000803 and (10.2) PZE-110000803 and PZE-110001270. The first maize plant can be crossed to a second maize plant, and the progeny resulting from the cross can be evaluated for the allele of the first maize plant. Progeny plants that possess the allele from the first maize plant can be selected as having decreased GLS. Maize plants selected by this method are also of interest.

### BRIEF DESCRIPTION OF FIGURES AND SEQUENCE LISTINGS

The invention can be more fully understood from the following detailed description and the accompanying drawings and Sequence Listing which form a part of this application. The Sequence Listing contains the one letter code for nucleotide sequence characters and the three letter codes for amino acids as defined in conformity with the IUPAC-IUBMB standards described in Nucleic Acids Research 13:3021-3030 (1985) and in the Biochemical Journal 219 (No. 2): 345-373 (1984) which are herein incorporated by reference in their entirety. The symbols and format used for nucleotide and amino acid sequence data comply with the rules set forth in 37 C.F.R. §1.822.

SEQ ID NOs: 1-32 are the marker assisted breeding (MAB) friendly markers identified within each chromosomal interval by the Single Donor vs. Elite Panel (SDvEP) method.

SEQ ID NOs: 10 and 33-78 are markers that define the 5′ and 3′ borders of the chromosomal intervals defined within.

### DETAILED DESCRIPTION OF THE INVENTION

The present invention provides methods for identifying and selecting maize plants with increased GLS resistance. The following definitions are provided as an aid to understand the invention.

The term “allele” refers to one of two or more different nucleotide sequences that occur at a specific locus.

An “amplicon” is amplified nucleic acid, e.g., a nucleic acid that is produced by amplifying a template nucleic acid by any available amplification method (e.g., PCR, LCR, transcription, or the like).

The term “amplifying” in the context of nucleic acid amplification is any process whereby additional copies of a selected nucleic acid for a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods. The term “assemble” applies to BACs and their propensities for coming together to form contiguous stretches of DNA. A BAC “assembles” to a contig based on sequence alignment, if the BAC is sequenced, or via the alignment of its BAC fingerprint to the fingerprints of other BACs. The assemblies can be found using the Maize Genome Browser, which is publicly available on the internet.

An allele is “associated with” a trait when it is linked to it and when the presence of the allele is an indicator that the desired trait or trait form will occur in a plant comprising the allele.

The “B73 reference genome, version 2” is the physical and genetic framework of the maize B73 genome. It is the result of a sequencing effort utilizing a minimal tiling path of approximately 19,000 mapped BAC clones, and focusing on producing high-quality sequence coverage of all identifiable gene-containing regions of the maize genome. These regions were ordered, oriented, and along with all of the intergenic sequences, anchored to the extant physical and genetic maps of the maize genome. It can be accessed using a genome browser, the Maize Genome Browser, that is publicly available on the internet that facilitates user interaction with sequence and map data.

A “BAC”, or bacterial artificial chromosome, is a cloning vector derived from the naturally occurring F factor of Escherichia coli. BACs can accept large inserts of DNA sequence. In maize, a number of BACs, or bacterial artificial chromosomes, each containing a large insert of maize genomic DNA, have been assembled into contigs (overlapping contiguous genetic fragments, or “contiguous DNA”).

“Backcrossing” refers to the process whereby hybrid progeny are repeatedly crossed back to one of the parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular Marker Data, pp. 41-43. The initial cross gives rise to the F1 generation: the term “BC1” then refers to the second use of the recurrent parent, “BC2” refers to the third use of the recurrent parent, and so on.

The term “causative allele” refers to an allele that is responsible for a particular phenotype.

A centimorgan (“cM”) is a unit of measure of recombination frequency. One cM is equal to a 1% chance that a marker at one genetic locus will be separated from a marker at a second locus due to crossing over in a single generation.

“Chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The term “chromosomal interval” designates any and all intervals defined by any of the markers set forth in this invention. Chromosomal intervals that correlate with increased GLS resistance are provided (e.g. the interval, located on chromosome 1, comprises and is flanked by PZE-101025686 and PZE-101026265).

The term “complement” refers to a nucleotide sequence that is complementary to a given nucleotide sequence, i.e., the sequences are related by the base-pairing rules.

The term “contiguous DNA” refers to overlapping contiguous genetic fragments.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

A “favorable allele” is the allele at a particular locus that confers, or contributes to, a desirable phenotype, e.g., increased GLS resistance, or alternatively, is an allele that allows the identification of plants with increased GLS susceptibility that can be removed from a breeding program or planting (“counter-selection”). A favorable allele of a marker is a marker allele that segregates with the favorable phenotype, or alternatively, segregates with the unfavorable plant phenotype, therefore providing the benefit of identifying plants.

“Fragment” is intended to mean a portion of a nucleotide sequence. Fragments can be used as hybridization probes or PCR primers using methods disclosed herein.

A “genetic map” is a description of genetic linkage relationships among loci on one or more chromosomes (or chromosomes) within a given species, generally depicted in a diagrammatic or tabular form. For each genetic map, distances between loci are measured by the recombination frequencies between them, and recombinations between loci can be detected using a variety of molecular genetic markers (also called molecular markers). A genetic map is a product of the mapping population, types of markers used, and the polymorphic potential of each marker between different populations. The order and genetic distances between loci can differ from one genetic map to another. However, information such as marker position and order can be correlated between maps by determining the physical location of the markers on the chromosome of interest, using the B73 reference genome, version 2, which is publicly available on the internet. One of ordinary skill in the art can use the publicly available genome browser to determine the physical location of markers on a chromosome.

The term “Genetic Marker” shall refer to any type of nucleic acid based marker, including but not limited to, Restriction Fragment Length Polymorphism (RFLP), Simple Sequence Repeat (SSR) Random Amplified Polymorphic DNA (RAPD), Cleaved Amplified Polymorphic Sequences (CAPS) (Rafalski and Tingey, 1993, Trends in Genetics 9:275-280), Amplified Fragment Length Polymorphism (AFLP) (Vos et al, 1995, Nucleic Acids Res. 23:4407-4414), Single Nucleotide Polymorphism (SNP) (Brookes, 1999, Gene 234:177-186), Sequence Characterized Amplified Region (SCAR) (Pecan and Michelmore, 1993, Theor. Appl. Genet, 85:985-993), Sequence Tagged Site (STS) (Onozaki et al. 2004, Euphytica 138:255-262), Single Stranded Conformation Polymorphism (SSCP) (Orita et al., 1989, Proc Natl Aced Sci USA 86:2766-2770). Inter-Simple Sequence Repeat (ISR) (Blair et al. 1999, Theor. Appl. Genet. 98:780-792), Inter-Retrotransposon Amplified Polymorphism (IRAP), Retrotransposon-Microsatellite Amplified Polymorphism (REMAP) (Kalendar et al., 1999, Theor. Appl. Genet 98:704-711), an RNA cleavage product (such as a Lynx tag), and the like.

“Genetic recombination frequency” is the frequency of a crossing over event (recombination) between two genetic loci. Recombination frequency can be observed by following the segregation of markers and/or traits following meiosis.

“Genome” refers to the total DNA, or the entire set of genes, carried by a chromosome or chromosome set.

“Genome-wide association study (GWAS)” is an examination of many common genetic variants (e.g. single nucleotide polymorphisms) in different individuals to see if any variant is associated with a trait.

The term “genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple led, or, more generally, the term genotype can be used to refer to an individual's genetic make-up for all the genes in its genome.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells that can be cultured into a whole plant.

The term “gray leaf spot” or “GLS” refers to a foliar fungal disease of maize. The etiolologic agents are Cercospora zeae-maydis and Cercospora zein. GLS usually causes discoloration of the leaves and lesions on the leaves.

A “haplotype” is the genotype of an individual at a plurality of genetic loci, i.e. a combination of alleles. Typically, the genetic loci described by a haplotype are physically and genetically linked, i.e., on the same chromosome segment. The term “haplotype” can refer to sequence, polymorphisms at a particular locus, such as a single marker locus, or sequence polymorphisms at multiple loci along a chromosomal segment in a given genome. The former can also be referred to as “marker haplotypes” or “marker alleles”, while the latter can be referred to as “long-range haplotypes”.

The “heritability (h²)” of a trait within a population is the proportion of observable differences in a trait between individuals within a population that is due to genetic differences. The h² value of the QTL is a percentage of variation that is explained by genetics, instead of environment.

A “heterotic group” comprises a set of genotypes that perform well when crossed with genotypes from a different heterotic group (Hallauer at al. (1998) Corn breeding, p. 463-564. In G. F. Sprague and J. W. Dudley (ed) Corn and corn improvement). Inbred lines are classified into heterotic groups, and are further subdivided into families within a heterotic group, based on several criteria such as pedigree, molecular marker-based associations, and performance in hybrid combinations (Smith at al. (1990) Theor. Appl. Gen. 80:833-840). The two most widely used heterotic groups in the United States are referred to as “Iowa Stiff Stalk Synthetic” (BSSS) and “Lancaster” or “Lancaster Sure Crop” (sometimes referred to as NSS, or Iron-Stiff Stalk).

The term “heterozygous” means a genetic condition wherein different alleles reside at corresponding loci on homologous chromosomes.

The term “homozygous” means a genetic condition wherein identical alleles reside at corresponding loci on homologous chromosomes.

“Hybridization” or “nucleic acid hybridization” refers to the pairing of complementary RNA and DNA strands as well as the pairing of complementary DNA single strands.

The term “hybridize” means the formation of base pairs between complementary regions of nucleic acid strands.

The term “indel” refers to an insertion or deletion, wherein one line may be referred to as having an insertion relative to a second line, or the second line may be referred to as having a deletion relative to the first line.

The term “introgression” or “introgressing” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background. For example, the chromosome 1 locus described herein may be introgressed into a recurrent parent that has problematic GLS. The recurrent parent line with the introgressed gene or locus then has decreased GLS.

As used herein, the term “linkage” is used to describe the degree with which one marker locus is associated with another marker locus or some other locus (for example, a GLS locus). The linkage relationship between a molecular marker and a phenotype is given as a “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units for cM). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10 (e.g., about 9%, 8%, 7%, 6%, 5%, 4%, 3%, 2%, 1%, 0.75%, 0.5%, 0.25%, or less) are also said to be “proximal to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

The term “linkage disequilibrium” refers to a non-random segregation of genetic loci or traits for both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same chromosome.) As used herein, linkage can be between two markers, or alternatively between a marker and a phenotype. A marker locus can be “associated with” (linked to) a trait, e.g., decreased GLS. The degree of linkage of a molecular marker to a phenotypic trait is measured, e.g. as a statistical probability of co-segregation of that molecular marker with the phenotype.

Linkage disequilibrium is most commonly assessed using the measure r², which is calculated using the formula described by Hill, W. G. and Robertson, A, Theor Appl. Genet 38:226-231 (1988). When r²=1, complete LD exists between the two marker loci, meaning that the markers have not been separated by recombination and have the same allele frequency. Values for r² above ⅓ indicate sufficiently strong LD to be useful for mapping (Ardlie at al., Nature Reviews Genetics 3:299-309 (2002)). Hence, alleles are in linkage disequilibrium when r² values between pairwise marker loci are greater than or equal to 0.33, 0.4, 0.5, 0.6, 0.7, 0.8, 0.9, or 1.0.

As used herein, “linkage equilibrium” describes a situation where two markers independently segregate, i.e., sort among progeny randomly. Markers that show linkage equilibrium are considered unlinked (whether or not they lie on the same chromosome).

The “logarithm of odds (LOD) value” or “LOD score” (Risch, Science 255:803-804 (1992)) is used in interval mapping to describe the degree of linkage between two marker loci. A LOD score of three between two markers indicates that linkage is 1000 times more likely than no linkage, while a LOD score of two indicates that linkage is 100 times more likely than no linkage. LOD scores greater than or equal to two may be used to detect linkage.

A “locus” is a position on a chromosome where a gene or marker is located.

“Maize” refers to a plant of the Zea mays L. ssp. mays and is also known as “corn”.

The term “maize plant” includes: whole maize plants, maize plant cells, maize plant protoplast, maize plant cell or maize tissue cultures from which maize plants can be regenerated, maize plant calli, and maize plant cells that are intact in maize plants or parts of maize plants, such as maize seeds, maize cobs, maize flowers, maize cotyledons, maize leaves, maize stems, maize buds, maize roots, maize root tips, and the like.

A “marker” is a nucleotide sequence or encoded product thereof (e.g., a protein) used as a point of reference. For markers to be useful at detecting recombinations, they need to detect differences, or polymorphisms, within the population being monitored. For molecular markers, this means differences at the DNA level due to polynucleotide sequence differences (e.g. SSRs, RFLPs, FLPs, SNPs). The genomic variability can be of any origin, for example, insertions, deletions, duplications, repetitive elements, point mutations, recombination events, or the presence and sequence of transposable elements. Molecular markers can be derived from genomic or expressed nucleic acids (e.g., ESTs) and can also refer to nucleic acids used as probes or primer pairs capable of amplifying sequence fragments via the use of PCR-based methods. A large number of maize molecular markers are known in the art, and are published or available from various sources, such as the Maize GDB Internet resource and the Arizona Genomics Institute Internet resource run by the University of Arizona.

Markers corresponding to genetic polymorphisms between members of a population can be detected by methods well-established in the art. These include, e.g., DNA sequencing, PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

A “marker allele”, alternatively an “allele of a marker locus”, can refer to one of a plurality of polymorphic nucleotide sequences found at a marker locus in a population that is polymorphic for the marker locus.

“Marker assisted selection” (or MAS) is a process by which phenotypes are selected based on marker genotypes.

“Marker assisted counter-selection” is a process by which marker genotypes are used to identify plants that will not be selected, allowing them to be removed from a breeding program or planting.

A “marker locus” is a specific chromosome location in the genome of a species where a specific marker can be found. A marker locus can be used to track the presence of a second linked locus, e.g., a linked locus that encodes or contributes to expression of a phenotypic trait. For example, a marker locus can be used to monitor segregation of alleles at a locus, such as a QTL or single gene, that are genetically or physically linked to the marker locus.

A “marker probe” is a nucleic add sequence or molecule that can be used to identify the presence of a marker locus, e.g., a nucleic acid probe that is complementary to a marker locus sequence, through nucleic add hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e. genotype) the particular allele that is present at a marker locus.

The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a via a plant without the insertion. Thus, the marker need only indicate whether the indel region is present or absent. Any suitable marker detection technology may be used to identify such a hybridization marker, e.g., SNP technology is used in the examples provided herein.

“Nucleotide sequence”, “polynucleotide”, “nucleic acid sequence”, and “nucleic acid fragment” are used interchangeably and refer to a polymer of RNA or DNA that is single- or double-stranded, optionally containing synthetic, non-natural or altered nucleotide bases. A “nucleotide” is a monomeric unit from which DNA or RNA polymers are constructed, and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate. “G” for guanylate or deoxyguanylate. “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “phenotype”, or “phenotypic trait” or “trait” refers to one or more traits of an organism. The phenotype can be observable to the naked eye, or by any other means of evaluation known in the art, e.g., microscopy, biochemical analysis, or an electromechanical assay. In some cases, a phenotype is directly controlled by a single gene or genetic locus, i.e., a “single gene trait”. In other cases, a phenotype is the result of several genes.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination.

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

A “polymorphism” is a variation in the DNA that is too common to be due merely to new mutation. A polymorphism must have a frequency of at least 1% in a population. A polymorphism can be a single nucleotide polymorphism, or SNP, or an insertion/deletion polymorphism, also referred to herein as an “indel”.

The “probability value” or “p-value” is the statistical likelihood that the particular combination of a phenotype and the presence or absence of a particular marker allele is random. Thus, the lower the probability score, the greater the likelihood that a phenotype and a particular marker will co-segregate. In some aspects, the probability score is considered “significant” or “nonsignificant”. In some embodiments, a probability score of 0.05 (p=0.05, or a 5% probability) of random assortment is considered a significant indication of co-segregation. However, an acceptable probability can be any probability of less than 50% (p=0.5). For example, a significant probability can be less than 0.25, less than 0.20, less than 0.15, less than 0.1, less than 0.05, less than 0.01, or less than 0.001.

The term “progeny” refers to the offspring generated from a cross.

A “progeny plant” is generated from a cross between two plants.

A “reference sequence” is a defined sequence used as a basis for sequence comparison. The reference sequence is obtained by genotyping a number of lines at the locus, aligning the nucleotide sequences in a sequence alignment program (e.g. Sequencher), and then obtaining the consensus sequence of the alignment.

The “Single Donor vs. Elite Panel (SDvEP)” method (as described in U.S. 61/700,427) has the potential to find a molecular marker under the QTL confidence interval that discriminates an allele which is present in a genome of a single donor variety that has a trait, and absent in genomes of varieties that do not have this trait.

A “single nucleotide polymorphism (SNP)” is a DNA sequence variation occurring when a single nucleotide—A, T, C or G—in the genome (or other shared sequence) differs between members of a biological species or paired chromosomes in an individual. For example, two sequenced DNA fragments from different individuals, AAGCCTA to AAGCTTA, contain a difference in a single nucleotide.

Sequence alignments and percent identity calculations may be determined using a variety of comparison methods designed to detect homologous sequences including, but not limited to, the MEGALIGN® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp, CABIOS. 5:151-153 (1989)) with the default parameters (GAP PENALTY=10, GAP LENGTH PENALTY=10), Default parameters for pairwise alignments and calculation of percent identity of protein sequences using the Clustal V method are KTUPLE=1, GAP PENALTY=3, WINDOW=5 and DIAGONALS SAVED=5. For nucleic adds these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Before describing the present invention in detail, it should be understood that this invention is not limited to particular embodiments. It also should be understood that the terminology used herein is for the purpose of describing particular embodiments, and is not intended to be limiting. As used herein and in the appended claims, terms in the singular and the singular forms “a”, “an” and “the”, for example, include plural referents unless the content clearly dictates otherwise. Thus, for example, reference to “plant”, “the plant” or “a plant” also includes a plurality of plants. Depending on the context, use of the term “plant” can also include genetically similar or identical progeny of that plant. The use of the term “a nucleic acid” optionally includes many copies of that nucleic acid molecule.

### Genetic Mapping

It has been recognized for quite some time that specific genetic loci correlating with particular phenotypes, such as increased GLS resistance, can be mapped in an organism's genome. The plant breeder can advantageously use molecular markers to identify desired individuals by detecting marker alleles that show a statistically significant probability of co-segregation with a desired phenotype, manifested as linkage disequilibrium. By identifying a molecular marker or clusters of molecular markers that co-segregate with a trait of interest, the breeder is able to rapidly select a desired phenotype by selecting for the proper molecular marker allele (a process called marker-assisted selection, or MAS).

A variety of methods well known in the art are available for detecting molecular markers or clusters of molecular markers that co-segregate with a trait of interest, such as reduced GLS. The basic idea underlying these methods is the detection of markers, for which alternative genotypes (or alleles) have significantly different average phenotypes. Thus, one makes a comparison among marker loci of the magnitude of difference among alternative genotypes (or alleles) or the level of significance of that difference. Trait genes are inferred to be located nearest the marker(s) that have the greatest associated genotypic difference.

Two such methods used to detect trait loci of interest are: 1) Population-based association analysis and 2) Traditional linkage analysis. In a population-based association analysis, lines are obtained from pre-existing populations with multiple founders, e.g. elite breeding lines. Population-based association analyses rely on the decay of linkage disequilibrium (LD) and the idea that in an unstructured population, only correlations between genes controlling a trait of interest and markers closely linked to those genes will remain after so many generations of random mating. In reality, most pre-existing populations have population substructure. Thus, the use of a structured association approach helps to control population structure by allocating individuals to populations using data obtained from markers randomly distributed across the genome, thereby minimizing disequilibrium due to population structure within the individual populations (also called subpopulations). The phenotypic values are compared to the genotypes (alleles) at each, marker locus for each line in the subpopulation. A significant marker-trait association indicates the dose proximity between the marker locus and one or more genetic loci that are involved in the expression of that trait.

The same principles underlie traditional linkage analysis; however, LD is generated by creating a population from a small number of founders. The founders are selected to maximize the level of polymorphism within the constructed population, and polymorphic sites are assessed for their level of cosegregation with a given phenotype. A number of statistical methods have been used to identify significant marker-trait associations. One such method is an interval mapping approach (Lander and Botstein, Genetics 121:185-199 (1989), in which each of many positions along a genetic map (say at 1 cM intervals) is tested for the likelihood that a gene controlling a trait of interest is located at that position. The genotype/phenotype data are used to calculate for each test position a LOD score (log of likelihood ratio). When the LOD score exceeds a threshold value, there is significant evidence for the location of a gene controlling the trait of interest at that position on the genetic map (which will fall between two particular marker loci).

Although the genetic mapping approaches described within offer high QTL detection power, resolution remains low due to inaccurate recombination information (Bennewitz et al. 2002). Several approaches can overcome the limitations of traditional QTL mapping and include genotyping-by-sequencing (GBS) and genome-wide association studies (GWAS). Advancements in next-generation sequencing (NGS) technology have provided an inexpensive means for whole genome sequencing and re-sequencing in many species. The availability of the technology has transformed the way genomes are sequenced, polymorphisms are discovered, and how populations are genotyped. GBS has been developed as a simple, but robust tool for association studies and genomics-assisted breeding in a range of species including those with complex genomes. GBS uses restriction enzymes for targeted complexity reduction followed by multiplex sequencing to produce high-quality polymorphism data at a relatively low per sample cost. As a result, GBS can provide an abundance of informative genome-wide and high-density markers for mapping. High-density markers can significantly improve the resolution of QTL mapping, facilitating the discovery of additional recombination events and exact recombination breakpoints. The flexibility of GBS in regards to species, populations, and research objectives makes this an ideal tool for plant genetics studies and the practice of applied plant breeding.

In addition to advances in NGS technology, mapping approaches using genome-wide association studies (GWAS) overcomes the limitations of traditional QTL mapping by providing higher resolution. GWAS uses a mapping population that is composed of genetically unrelated individuals with unknown pedigrees in order to examine many common genetic variants to determine if any variant is associated with a trait. The advent of high-density SNP genotyping allowed whole-genome scans to identify often small haplotype blocks that are significantly correlated with quantitative trait variation. These approaches have enabled recent plant studies that have been successful in identifying loci that explain large portions of phenotypic variation.

Markers Associated with Gray Leaf Spot Resistance

Markers associated with GLS resistance are identified herein. The methods involve detecting the presence of at least one marker allele associated with the enhanced resistance in the germplasm of a maize plant. The marker locus can be selected from any of the marker loci provided in Table 2, including chr1_{—}15269379, PZE-101188909, chr2_{—}6858691, PZE-102041193, PZE-102072013, chr2_{—}44697986, PZE-102079279, PZE-102088902, PZE-103053562, PZE-104093278, Chr4_{—}180264145, PZE-105165816, PZE-106100504, PZE-106107639, PZE-107004786, PZE-107020739, chr8_{—}7675588, PZE-108020413, PZE-108022834, PZE-108047366, GLS_chr8_{—}80296742, GLS_chr8_{—}80499765, PZE-108048175, PZE-108048978, GLS_chr8_{—}83335579, GLS_chr8_{—}86463733, GLS_chr8_{—}87640198, PZE-108050255, PZE-109017122, PZE-109084575, PZE-110000028, PZE-110000899, and any other marker linked to these markers (linked markers can be determined from the Maize GDB resource).

The genetic elements or genes located on a contiguous linear span of genomic DNA on a single chromosome are physically linked. Interval markers described in Table 1 are highly associated with GLS resistance, and delineate GLS resistance QTL. Any polynucleotide that assembles to the contiguous DNA between and including SEQ ID NOs: 10 and 33-55 (the reference sequences for the 5′ interval markers), or a nucleotide sequence that is 95% identical to SEQ ID NOs: 10 and 33-55 based on the Clustal V method of alignment, and SEQ ID NOs: 55-78 (the reference sequences for 3′ interval markers), or a nucleotide sequence that is 95% identical to SEQ ID NOs: 55-78 based on the Clustal V method of alignment, can house marker loci that are associated with GLS resistance.

A common measure of linkage is the frequency with which traits cosegregate. This can be expressed as a percentage of cosegregation (recombination frequency) or in centiMorgans (cM). The cM is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency.

Marker loci are themselves traits and can be assessed according to standard linkage analysis by tracking the marker loci during segregation. Thus, one cM is equal to a 1% chance that a marker locus will be separated from another locus, due to crossing over in a single generation.

Other markers linked to the markers listed in Tables 1 and 2 can be used to predict GLS resistance in a maize plant. This includes any marker within 50 cM of SEQ ID NOs: 1-78, the markers associated with the GLS resistance. The closer a marker is to a gene controlling a trait of interest, the more effective and advantageous that marker is as an indicator for the desired trait. Closely linked loci display an inter-locus cross-over frequency of about 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus) display a recombination frequency of about 1% or less, e.g., about 0.75% or less, more preferably about 0.5% or less, or yet more preferably about 0.25% or less. Thus, the loci are about 10 cM, 9 cM, 8 cM, 7 cM, 6 cM, 5 cM, 4 cM, 3 cM, 2 cM, 1 cM, 0.75 cM, 0.5 cM or 0.25 cM or less apart. Put another way, two loci that are localized to the same chromosome, and at such a distance that recombination between the two loci occurs at a frequency of less than 10% (e.g., about 9%, 8% 7%, 6%, 5%, 4%, 3%, 2% 1%, 0.75%, 0.5%, 0.25.degree, or less) are said to be “proximal to” each other.

Although particular marker alleles can show co-segregation with increased GLS resistance, it is important to note that the marker locus is not necessarily responsible for the expression of the GLS resistance phenotype. For example, it is not a requirement that the marker polynucleotide sequence be part of a gene that imparts increased GLS resistance (for example, be part of the gene's open reading frame). The association between a specific marker allele and the increased GLS resistance phenotype is due to the original “coupling” linkage phase between the marker allele and the allele in the ancestral maize line from which the allele originated. Eventually, with repeated recombination, crossing over events between the marker and genetic locus can change this orientation. For this reason, the favorable marker allele may change depending on the linkage phase that exists within the resistant parent used to create segregating populations. This does not change the fact that the marker can be used to monitor segregation of the phenotype. It only changes which marker allele is considered favorable in a given segregating population.

The term “chromosomal interval” designates any and all intervals defined by any of the markers set forth in this invention. Chromosomal intervals that correlate with GLS resistance are provided. These intervals, located on chromosomes 1-10, comprise and are flanked by 5′ and 3′ interval markers SEQ ID NOs: 33 and 56; 34 and 57; 35 and 58; 36 and 59; 37 and 60; 38 and 61; 39 and 62; 40 and 63; 41 and 64; 10 and 65; 42 and 66; 43 and 67; 44 and 68; 45 and 69; 46 and 70; 47 and 71; 48 and 72; 49 and 73; 50 and 74; 51 and 75; 52 and 76; 53 and 77; 54 and 55; and 55 and 78.

A variety of methods well known in the art are available for identifying chromosomal intervals. The boundaries of such chromosomal intervals are drawn to encompass markers that will be linked to the gene controlling the trait of interest. In other words, the chromosomal interval is drawn such that any marker that lies within that interval (including the terminal markers that define the boundaries of the interval) can be used as a marker for GLS resistance. The intervals described above encompass a cluster of markers that co-segregate with GLS resistance. The clustering of markers occurs in relatively small domains on the chromosomes, indicating the presence of a gene controlling the trait of interest in those chromosome regions. The intervals were drawn to encompass the markers that co-segregate with GLS resistance. The intervals encompass markers that map within the intervals as well as the markers that define the termini. For example, PZE-101025686 and PZE-101026265, separated by 633,298 bp based on the B73 reference genome, version 2, define a chromosomal interval encompassing a cluster of markers that co-segregate with GLS resistance. An interval described by the terminal markers that define the endpoints of the interval will include the terminal markers and any marker localizing within that chromosomal domain, whether those markers are currently known or unknown.

Chromosomal intervals can also be defined by markers that are linked to (show linkage disequilibrium with) a marker of interest, and is a common measure of linkage disequilibrium (LD) in the context of association studies. If the r² value of LD between any chromosome 1 marker locus lying within the interval of PZE-101025686 and PZE-101026265, and an identified marker within that interval that has an allele associated with increased GLS resistance is greater than ⅓ (Ardlie et al. Nature Reviews Genetics 3:299-309 (2002)), the loci are linked.

A marker of the invention can also be a combination of alleles at marker loci, otherwise known as a haplotype. The skilled artisan would expect that there might be additional polymorphic sites at marker loci in and around the markers identified herein, wherein one, or more polymorphic sites is in linkage disequilibrium (LD) with an allele associated with increased GLS resistance. Two particular alleles at different polymorphic sites are said to be in LD if the presence of the allele at one of the sites tends to predict the presence of the allele at the other site on the same chromosome (Stevens, Mol. Diag. 4:309-17 (1999)).

Single Donor Vs. Elite Panel Method

The Single Donor vs. Elite Panel (SDvEP) method has the potential to find a molecular marker under the QTL chromosome interval that discriminates an allele which is present in a genome of a single donor variety with a trait of interest, and absent in genomes of varieties that do not have this trait. The main concept of this method is an assumption that a causative mutation controlling a trait is evolutionary conserved in a donor line(s) and absent in unrelated elite lines which explains the lack of a trait in those line. A marker identified by SDvEP method might or might not represent the causative mutation though. However, a marker will (1) be significantly associated with a trait (2) at least detect an allele that is a characteristic of a donor line only and (3) can be easily tracked in segregating populations without a fear of selecting false positive plants. A marker detected by this method is called a marker-assisted breeding (MAB) friendly marker. This method is ideal for the traits which are controlled by a single gene or by major QTL and several minor QTL. This method has no value if the trait is controlled epigenetically, which assumes no structural variations. In an embodiment, SDvEP resolves the phenotype to specific loci, a single locus, or even a single nucleotide.

MAB friendly markers identified using the SDvEP method are provided. A single MAB friendly marker was identified for each of the chromosomal intervals on chromosomes 1-10, as described within. The MAB friendly markers are set forth in Table 2.

### Marker Assisted Selection

Molecular markers can be used in a variety of, plant breeding applications (e.g. see Staub et al. (1996) Hortscience 729-741; Tanksley (1983) Plant Molecular Biology Reporter 1: 3-8). One of the main areas of interest is to increase the efficiency and reliability of selecting genotypes with a trait of interest through marker-assisted selection (MAS). A molecular marker that demonstrates linkage with a locus affecting a desired phenotypic trait provides a useful tool for the selection of the trait in a plant population. This is particularly true when the phenotype is hard to assay, e.g. many quantitatively inherited disease resistance traits, or, occurs at a late stage in plant development, e.g. kernel characteristics, or, is environmentally dependent, e.g. seed quality traits. Since DNA marker assays are less laborious and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing the trait, which can result in false positives. Having flanking markers decreases the chances that false positive selection will occur as a double recombination event would be needed. The ideal situation is to have a marker in the gene itself, so that recombination cannot occur between the marker and the gene. Such a marker is called a ‘perfect marker’.

When a gene is introgressed by MAS, it is not only the gene that is introduced but also the flanking regions (Gepts. (2002). Crop Sci; 42: 1780-1790). This is referred to as “linkage drag.” In the case where the donor plant is highly unrelated to the recipient plant, these flanking regions carry additional genes that may code for agronomically undesirable traits. This “linkage drag” may also result in reduced yield or other negative agronomic characteristics even after multiple cycles of backcrossing into the elite maize line. This is also sometimes referred to as “yield drag.” The size of the flanking region can be decreased by additional backcrossing, although this is not always successful, as breeders do not have control over the size of the region or the recombination breakpoints (Young et al, (1998) Genetics 120:579-585). In classical breeding it is usually only by chance that recombinations are selected that contribute to a reduction in the size of the donor segment (Tanksley et al. (1989). Biotechnology 7: 257-264). Even after 20 backcrosses in backcrosses of this type, one may expect to find a sizeable piece of the donor chromosome still linked to the gene being selected. With markers however, it is possible to select those rare individuals that have experienced recombination near the gene of interest. In 150 backcross plants, there is a 95% chance that at least one plant will have experienced a crossover within 1 cM of the gene, based on a single meiosis map distance. Markers will avow unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with, markers, while it would have required on average 100 generations without markers (See Tanksley et al., supra). When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

The availability of the B73 reference genome, version 2 and the integrated linkage maps of the maize genome containing increasing densities of public maize markers, has facilitated maize genetic mapping and MAS. See, e.g. the IBM2 Neighbors maps, which are available online on the Maize GDB website.

The key components to the implementation of MAS are (i) Defining the population within which the marker-trait association will be determined, which can be a segregating population, or a random or structured population; (ii) monitoring the segregation or association of polymorphic markers relative to the trait, and determining linkage or association using statistical methods; (iii) defining a set of desirable markers based on the results of the statistical analysis, and (iv) the use and/or extrapolation of this information to the current set of breeding germplasm to enable marker-based selection decisions to be made. The markers described in this disclosure, as well as other marker types such as SSRs and FLPs (such as RFLPs and AFLPs), can be used in marker assisted selection protocols.

SSRs can be defined as relatively short runs of tandemly repeated DNA with lengths of 6 bp or less (Tautz (1989) Nucleic Acid Research 17: 6463-6471; Wang et al. (1994) Theoretical and Applied Genetics, 88:1-6) Polymorphisms arise due to variation in the number of repeat units, probably caused by slippage during DNA replication (Levinson and Gutman (1987) Mol Biol Evol 4: 203-221). The variation in repeat length may be detected by designing PCR primers to the conserved non-repetitive flanking regions (Weber and May (1989) Am J Hum Genet. 44:388-396), SSRs are highly suited to mapping and MAS as they are multi-allelic, codominant, reproducible and amenable to high throughput automation (Rafalski et al. (1996) Generating and using DNA markers in plants. In Non-mammalian genomic analysis: a practical guide. Academic Press, pp 75-135).

Various types of SSR markers can be generated, and SSR profiles from resistant lines can be obtained by gel electrophoresis of the amplification products. Scoring of marker genotype is based on the size of the amplified fragment. An SSR service for maize is available to the public on a contractual basis by DNA Landmarks in Saint-Jean-sur-Richelieu, Quebec, Canada.

Various types of FLP markers can also be generated. Most commonly, amplification primers are used to generate fragment length polymorphisms. Such FLP markers are in many ways similar to SSR markers, except that the region amplified by the primers is not typically a highly repetitive region. Still, the amplified region, or amplicon, will have sufficient variability among germplasm, often due to insertions or deletions, such that the fragments generated by the amplification primers can be distinguished among polymorphic individuals, and such indels are known to occur frequently in maize (Bhattramakki et al. (2002). Plant Mol Biol 48, 539-547; Rafalski (2002b), supra).

SNP markers detect single base pair nucleotide substitutions. Of all the molecular marker types, SNPs are the most abundant, thus having the potential to provide the highest genetic map resolution (Bhattramakki et al. 2002 Plant Molecular Biology 48:539-547). SNPs can be assayed at an even higher level of throughput than SSRs, in a so-called ‘ultra-high-throughput’ fashion, as they do not require large amounts of DNA and automation of the assay may be straight-forward. SNPs also have the promise of being relatively low-cost systems. These three factors together make SNPs highly attractive for use in MAS. Several methods are available for SNP genotyping, including but not limited to, hybridization, primer extension, oligonucleotide ligation, nuclease cleavage, minisequencing and coded spheres. Such methods have been reviewed in: Gut (2001) Hum Mutat 17 pp, 475-492: Shi (2001) Clin Chem 47, pp. 164-172; Kwok (2000) Pharmacogenomics 1, pp. 95-100: Bhattramakki and Rafalski (2001) Discovery and application of single nucleotide polymorphism markers in plants. In: R, J Henry, Ed, Plant Genotyping: The DNA Fingerprinting of Plants, CABI Publishing, VVallingford. A wide range of commercially available technologies utilize these and other methods to interrogate SNPs including Masscode™. (Qiagen), Invader® (Third Wave Technologies), SnapShot® (Applied Biosystems), Taqman® (Applied Biosystems) and Beadarrays™ (Illumina).

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes can be more informative than, single SNPs and can be more descriptive of any particular genotype. For example, single SNP may be allele ‘T’ for a specific line or variety with increased GLS resistance, but the allele ‘T’ might also occur in the maize breeding population being utilized for recurrent parents. In this case, a haplotype, e.g. a combination of alleles at linked SNP markers, may be more informative. Once a unique haplotype has been assigned to a donor chromosomal region, that haplotype can be used in that population or any subset thereof to determine whether an individual has a particular gene. See, for example, WO2003054229. Using automated high throughput marker detection platforms known to those of ordinary skill in the art makes this process highly efficient and effective.

The sequences listed in Tables 1 and 2 can be readily used to obtain additional polymorphic SNPs (and other markers) within the QTL chromosome intervals listed in this disclosure. Markers within the described map regions can be hybridized to BACs or other genomic libraries, or electronically aligned with genome sequences, to find new sequences in the same approximate location as the described markers.

In addition to SSR's, FLPs and SNPs, as described above, other types of molecular markers are also widely used, including but not limited to markers derived from expressed sequence tags (ESTs), randomly amplified polymorphic DNA (RAPD), and other nucleic acid based markers.

Isozyme profiles and linked morphological characteristics can, in some cases, also be indirectly used as markers. Even though they do not directly detect DNA differences, they are often influenced by specific genetic differences. However, markers that detect DNA variation are far more numerous and polymorphic than isozyme or morphological markers (Tanksley (1983) Plant Molecular Biology Reporter 1:3-8).

Sequence alignments or contigs may also be used to find sequences upstream or downstream of the specific markers listed herein. These new sequences, close to the markers described herein, are then used to discover and develop functionally equivalent markers. For example, different physical and/or genetic maps are aligned to locate equivalent markers not described within this disclosure but that are within similar regions. These maps may be within the maize species, or even across other species that have been genetically or physically aligned with maize, such as rice, wheat, barley or sorghum.

In general, MAS uses polymorphic markers that have been identified as having a significant likelihood of co-segregation with GLS resistance. Such markers are presumed to map near a gene or genes that give the plant its GLS resistance phenotype, and are considered indicators for the desired trait, or markers. Plants are tested for the presence of a desired allele in the marker, and plants containing a desired genotype at one or more loci are expected to transfer the desired genotype, along with a desired phenotype, to their progeny. The means to identify maize plants that have increased GLS resistance by identifying plants that have a specified allele at any one of marker loci described herein, including SEQ ID NOs: 1-78 are presented herein.

The interval presented herein finds use in MAS to select plants that demonstrate increased GLS resistance. Any marker that maps within the chromosome intervals described within can be used for this purpose. In addition, haplotypes comprising alleles at one or more marker loci within the chromosome intervals described within can be used to introduce increased GLS resistance into maize lines or varieties. Any allele or haplotype that is in linkage disequilibrium with an allele associated with increased GLS resistance can be used in MAS to select plants with increased GLS resistance.

### EXAMPLES

The following examples are offered to illustrate, but not to limit, the appended claims. It is understood that the examples and embodiments described herein are for illustrative purposes only and that persons skilled in the art will recognize various reagents or parameters that can be altered without departing from the spirit of the invention or the scope of the appended claims.

Mapping QTL controlling GLS resistance resulted in the identification of 32 QTL across all ten chromosomes. QTL detection was performed using the GWAS approach. GWAS per se does not define the actual QTL intervals, it identifies markers linked to QTL. However, any marker that is co-segregating with the marker detected by GWAS will still give the same genetic information as the latter. That is why the block of co-segregating markers including the one that was detected by GWAS as a landmark linked to GLS resistance QTL were considered as a QTL interval in this study. The QTL intervals are described in Table 1. Many SNP markers were located under the QTL intervals in each chromosome. Theoretically, any of the markers could be considered genetically linked to the trait; however practically, not all of them are useful for MAS because they might discriminate alleles that are present both in the resistant line and other susceptible lines. A set of GLS resistant donor lines and 71 unrelated GLS susceptible inbred lines were genotyped by SNP markers located within the QTL intervals and assessed by the SDvEP method to identify MAB-friendly markers that would discriminate the alleles which are present in the GLS resistant lines (not necessarily in all) and absent in the genomes of the entire panel of 71 GLS susceptible elite lines.

### Example 1

### Genetic Materials

A Diversity Panel of ˜300 maize inbred lines, comprised of DAS proprietary corn germplasm and public lines of North and South American and African origin, was developed and used to carry out GWAS.

Genomic DNA samples for SNP genotyping were isolated from the lyophilized leaf tissue of DH individuals and maize inbred lines using a Qiagen DNA extraction kit (Qiagen, Valencia, Calif.) per manufacturer's instructions.

### Example 2

### Cercospora zeae-maydis Inoculation

Both liquid and dry application methods were used to inoculate the plants with Cercospora zeae-maydis. For liquid application, C. zeae-maydis was grown in CZ shake media [0.6 mM carboxymethyl cellulose (CMC), 7.3 mM KH2PO4, 0.06 M CaCO3 and 40% V8 Juice (Campbell Soup Co., Camden, N.J.)]. The shake cultures were mixed 1:1 with reverse osmosis water (roH2O) and ground using a blender until the stromata balls were ground into fine particles. The ground solution was poured through 4 layers of cheesecloth or 1 layer of washed unbleached muslin cloth. The solution was mixed 1:1 into a prepared 5 mg/L solution of CMC. The final solution was applied to the plants using a hand sprayer. For dry inoculation, the concentrated CZ shake culture was mixed with 250 ml of sterile roH2O and then 30 ml of the mixture was added to each 2.2 lb sterilized dry oat bag. Once the culture sporulated on the oats (approximately 14 days), the colonized oats were removed from the tray, dried for three days, and then ground. Approximately 0.08 gram of inoculum was deposited down the whorl of each corn plant to inoculate. Plants were inoculated with both methods twice, 7 days apart.

### Example 3

### Phenotypic Data Collection

The Diversity Panel was planted in two locations, Mount Vernon, Ind. (MV) and Davenport, Iowa (DAV) in the spring of 2011 and 2012. Fifteen kernels per line were planted in a single row. In each environment a GLS rating was conducted at least two times with the first rating taken immediately after flowering. In MV, the phenotypic data was collected twice, at 39 and 53 days after inoculation. In DAV, GLS was also rated twice, at 38 and 67 days after inoculation. Depending on the type of GLS resistance, corn responds differently to a pathogen: rectangular necrotic lesions are characteristics of susceptible lines, flecks are indicative of resistance, while chlorotic lesions with orange or yellow borders are characteristics of intermediate resistance.

Biological indices were assigned to each type of lesion (LTI): necrotic lesions—0.75, chlorotic lesion—0.20 and fleck—0.05. The second parameter taken into consideration was the percentage of infected area of a leaf covered by predominant lesion type (PLS). This was rated on a 1 (3-9% of infected leaf area) to 9 (>89% of infected leaf area) scale. Lesion type and infection spread were measured on three leaves per plant: at the leaf below the ear, the ear leaf and the leaf above the ear. To calculate the overall GLS severity of one plant per rating, the following formula was used: GS=[(LTI_{BE}*PLS_{BE})+(LTI_{EL}*PLS_{EL})+(LTI_{AE}*PLS_{AE})]/3, where LTI is the lesion type index, PLS is the predominant lesion spread, and BE, EL and AE are below ear leaf, ear level leaf and above ear leaf, respectively. Depending on the number of ratings per environment, the Area Under Disease Progress Curve (AUDPC) was calculated (Campbell and Madden, 1990), which represented the final phenotype. A resistant phenotype is associated with a lower AUDPC value.

### Example 4

### Molecular Analysis

The Diversity Panel was genotyped with the custom Infinium iSelects (Illumina, San Diego, Calif.), which consisted of 33,000 (33K) attempted bead types. The 33K iSelect consisted of gene-based SNPs evenly distributed across all ten maize chromosomes. Genotyping with the iSelect was performed using the BeadArray SNP genotyping platform and Infinium chemistry (Illumina, San Diego, Calif.) according to the manufacturer's protocols.

### Example 5

### QTL Analysis

A DAS proprietary model was used to implement GWAS. Table 1 summarizes the information about the locations of the QTL chromosome intervals identified. Based on analysis, 32 QTL were identified across all 10 chromosomes. Multiple QTL were identified on chromosomes 1, 2, 4, 6, 7, 8, 9, and 10.

As all SNP markers representing Infinium custom iSelect were previously mapped in a DAS internal genetic consensus map, genetic linkage blocks representing SNP markers linked to GLS resistance QTL and other SNPs co-segregating with the formers were identified. Based on the physical position of the extreme left and right markers representing those linkage blocks, putative QTL intervals were identified and presented in Table 1. As all those markers co-segregate, they represent one recombination block and carry identical genetic information.

TABLE 1
QTL intervals for GLS resistance.

| | Chromosome | Interval physical position | Marker delimiting 5′ | SEQ ID | Marker delimiting 3′ | SEQ ID |
|---|---|---|---|---|---|---|
| Chr | interval no. | (bp) | border of interval | NO. | border of interval | NO. |
| 1 | 1.1 | 15,173,493-15,806,791 | PZE-101025686 | 33 | PZE-101026265 | 56 |
| 1 | 1.2 | 232,742,869-241,372,571 | DAS-PZ-14748 | 34 | bz2-2 | 57 |
| 2 | 2.1 | 5,866,676-6,597,252 | PZE-102013511 | 35 | DAS-PZ-32659 | 58 |
| 2 | 2.2 | 20,400,259-20,716,246 | PZE-102040682 | 36 | Mo17-12859 | 59 |
| 2 | 2.3 | 48,588,699-51,329,892 | PZE-102070420 | 37 | Mo17-13313 | 60 |
| 2 | 2.4 | 52,559,203-53,617,879 | PZE-102072947 | 38 | PZE-102073407 | 61 |
| 2 | 2.5 | 60,913,205-62,728,758 | PZE-102078235 | 39 | PZE-102079631 | 62 |
| 2 | 2.6 | 86,787,579-127,444,590 | PZE-102088257 | 40 | PZE-102103382 | 63 |
| 3 | 3 | 58,903,070-73,647,198 | PZE-103052576 | 41 | PZE-103057593 | 64 |
| 4 | 4.1 | 169,618,397-170,650,398 | PZE-104093278 | 10 | DAS-PZ-8846 | 65 |
| 4 | 4.2 | 181,229,828-181,428,424 | DSDS0099-1 | 42 | PZE-104105141 | 66 |
| 5 | 5 | 209,721,807-209,867,696 | PZE-105166071 | 43 | DAS-PZ-14276 | 67 |
| 6 | 6.1 | 153,217,431-153,787,631 | DAS-PZ-18055 | 44 | PZE-106101510 | 68 |
| 6 | 6.2 | 156,882,692-157,028,535 | Mo17-12530 | 45 | Mo17-14401 | 69 |
| 7 | 7.1 | 3,074,024-3,169,036 | PZE-107004762 | 46 | PZE-107004893 | 70 |
| 7 | 7.2 | 19,090,361-20,247,641 | DAS-PZ-11250 | 47 | PHM4080.15 | 71 |
| 8 | 8.1 | 6,124,253-6,480,774 | PZE-108006063 | 48 | PZE-108006412 | 72 |
| 8 | 8.2 | 19,128,826-19,551,679 | PZE-108020151 | 49 | PZE-108020416 | 73 |
| 8 | 8.3 | 21389738-22,173,786 | PZE-108022528 | 50 | PZE-108023337 | 74 |
| 8 | 8.4-8.12 | 79,076,065-90,577,326 | PZE-108047170 | 51 | PZE-108051324 | 75 |
| 9 | 9.1 | 17,086,313-17,471,980 | PZE-109016836 | 52 | PZE-109017324 | 76 |
| 9 | 9.2 | 132,724,865-133,626,016 | PZE-109083580 | 53 | PZE-109084648 | 77 |
| 10 | 10.1 | 0-1,726,403 | PZE-110000036 | 54 | PZE-110000803 | 55 |
| 10 | 10.2 | 1,726,403-1,877,616 | PZE-110000803 | 55 | PZE-110001270 | 78 |

### Example 6

### Single Donor Vs. Elite Panel (SDvEP)

Because a QTL interval can be very broad and harbor many markers, the SDvEP method allows for the identification of the loci (alleles) that are evolutionary preserved in a donor line(s) and absent in all susceptible elite lines. The method is used to narrow down the QTL confidence interval and identify marker-assisted breeding (MAB) friendly markers. This methodology is based on mining of all polymorphisms located within the QTL interval and then comparing them between a single or several resistant lines (sources of resistance) and a large panel of lines susceptible to this disease. The rationale is that a causative allele must be present only in the resistant lines and never in the susceptible panel. The higher the depth of a susceptible panel, the more powerful is the trait-marker association. SDvEP method does not require any statistical treatment because it is based on presence/absence of a donor allele among elite lines.

For this study, an elite panel was comprised of 71 GLS susceptible lines, which were susceptible in all four environments (two years×two locations). The SDvEP method was applied to discover SNP markers within each QTL chromosomal interval that discriminate alleles putatively conserved in the GLS resistant lines and completely absent in GLS susceptible panel of 71 inbred lines. Table 1 shows MAB-friendly markers for each QTL chromosomal interval that were identified using the SDvEP method, as well as the underlying SNP and position within the chromosomal interval.

As a result of SDvEP analysis, 32 markers (1 marker per chromosome interval) were identified as MAB-friendly markers (Table 2). The specific SNP for each marker is included in the table, with the resistant allele underlined. Nine separate MAB-friendly markers were identified within one chromosomal interval, defined by PZE-108047170 and PZE-108051324, on chromosome 8. The markers within this interval include PZE-108047366, GLS_chr8_{—}80296742, GLS_chr8_{—}80499765, PZE-108048175, PZE-108048978, GLS_chr8_{—}83335579, GLS_chr8_{—}86463733, GLS_chr8_{—}87640198, and PZE-108050255. Since the SDvEP method identified all of these markers within the one interval as reliable markers for MAB, each marker could be considered to represent a unique QTL within this chromosome interval. Thus, the SNP markers within the QTL interval spanning from PZE-108047170 to PZE-108051324 might represent a complex locus that consists of at least nine genes controlling GLS resistance. The specific SNP for each marker is described in Table 2, with the resistant allele coming from different GLS resistant lines underlined.

TABLE 2
Marker assisted breeding friendly markers for each
QTL chromosome interval based on SDvEP method.

| | Chromosome | | SEQ | | |
|---|---|---|---|---|---|
| | interval | MAB-friendly | ID | | Position |
| Chr | no. | marker | NO. | SNP | (bp) |
| 1 | 1.1 | chr1_15269379 | 1 | [T/G] | 15,269,379 |
| 1 | 1.2 | PZE-101188909 | 2 | [A/G] | 233,651,768 |
| 2 | 2.1 | chr2_6858691 | 3 | [A/G] | 5,924,858 |
| 2 | 2.2 | PZE-102041193 | 4 | [A/G] | 20,608,418 |
| 2 | 2.3 | PZE-102072013 | 5 | [T/C] | 51,239,296 |
| 2 | 2.4 | chr2_44697986 | 6 | [T/C] | 53,616,458 |
| 2 | 2.5 | PZE-102079279 | 7 | [A/G] | 62,099,369 |
| 2 | 2.6 | PZE-102088902 | 8 | [T/G] | 88,613,256 |
| 3 | 3 | PZE-103053562 | 9 | [T/G] | 60,573,890 |
| 4 | 4.1 | PZE-104093278 | 10 | [T/C] | 169,618,397 |
| 4 | 4.2 | Chr4_180264145 | 11 | [T/C] | 180,264,146 |
| 5 | 5 | PZE-105165816 | 12 | [T/A] | 209,732,639 |
| 6 | 6.1 | PZE-106100504 | 13 | [T/C] | 153,414,853 |
| 6 | 6.2 | PZE-106107639 | 14 | [A/G] | 156,924,799 |
| 7 | 7.1 | PZE-107004786 | 15 | [T/C] | 3,074,900 |
| 7 | 7.2 | PZE-107020739 | 16 | [A/G] | 19,500,572 |
| 8 | 8.1 | chr8_7675588 | 17 | [A/G] | 6,253,558 |
| 8 | 8.2 | PZE-108020413 | 18 | [T/C] | 19,550,800 |
| 8 | 8.3 | PZE-108022834 | 19 | [A/G] | 21,810,604 |
| 8 | 8.4 | PZE-108047366 | 20 | [A/G] | 79,424,520 |
| 8 | 8.5 | GLS_chr8_80296742 | 21 | [T/C] | 80,222,900 |
| 8 | 8.6 | GLS_chr8_80499765 | 22 | [T/G] | 80,389,467 |
| 8 | 8.7 | PZE-108048175 | 23 | [T/C] | 81,163,985 |
| 8 | 8.8 | PZE-108048978 | 24 | [A/G] | 82,523,744 |
| 8 | 8.9 | GLS_chr8_83335579 | 25 | [T/C] | 83,246,299 |
| 8 | 8.1 | GLS_chr8_86463733 | 26 | [T/C] | 85,845,207 |
| 8 | 8.11 | GLS_chr8_87640198 | 27 | [A/G] | 87,497,214 |
| 8 | 8.12 | PZE-108050255 | 28 | [T/G] | 87,676,974 |
| 9 | 9.1 | PZE-109017122 | 29 | [T/G] | 17,232,395 |
| 9 | 9.2 | PZE-109084575 | 30 | [A/G] | 133,586,192 |
| 10 | 10.1 | PZE-110000028 | 31 | [T/A] | 123,712 |
| 10 | 10.2 | PZE-110000899 | 32 | [T/C] | 1,877,616 |

Closely linked markers flanking the locus of interest that have alleles in linkage disequilibrium with a favorable allele at that locus may be effectively used to select for progeny plants with increased GLS resistance. Thus, the markers described in herein, such as those listed in Tables 1 and 2, as well as other markers genetically or physically mapped to the same chromosomal intervals, may be used to select for maize plants with increased GLS resistance. Typically, a set of these markers will be used (e.g. 2 or more, 3 or more, 4 or more, 5 or more) in the regions flanking the locus of interest. Optionally, a marker within the actual gene and/or locus may be used.

### REFERENCES

Aranzana M J, Kim S, Zhao K, Bakker E, Horton M, Jakob K, Lister C, . . . and Nordborg M (2005) Genome-wide association mapping in Arabidopsis identifies previously known flowering time and pathogen resistance genes. PLoS Genetics, 1(5), e60.
Balint-Kurti P J, Wisser R and Zwonitzer J C (2008) Use of an advanced intercross line population for precise mapping of quantitative trait loci for gray leaf spot resistance in Maize. Crop Sci. 48: 1696-1704.
Bennewitz J, Reinsch N, and Kalm E (2002) Improved confidence intervals in quantitative trait loci mapping by permutation bootstrapping. Genetics, 160(4), 1673-1686.
Bubeck D M, Goodman M M, Beavis W D, and Grant D (1993) Quantitative trait loci controlling resistance to gray leaf spot in maize. Crop Science, 33(4), 838-847.
Campbell C L and Madden L V (1990) Temporal analysis of epidemics. I: Description and comparison of disease progress curves. p. 161-202. In Introduction to plant disease epidemiology. John Wiley & Sons. N.Y.
Coates S T and White D G (1998) Inheritance of resistance to gray leaf spot in crosses involving selected resistant inbred lines of corn. Phytopathology, 88(9), 972-982.
Donahue P J, Stromberg E L, and Myers S L (1991) Inheritance of reaction to gray leaf spot in a diallel cross of 14 maize inbreds. Crop Science, 31(4), 926-931.
Elshire R J, Glaubitz J C, Sun Q, Poland J A, Kawamoto K, Buckler E S, Mitchell S E (2011) A robust, simple genotyping-by-sequencing (GBS) approach for high diversity species. PLOS One: 6:e19379.
Gevers H O, Lake J K (1994) Diallel cross analysis of resistance to gray leaf spot in maize. Plant Dis 78: 379-383.
Gordon S G, Lipps P E, Pratt R C (2006) Heritability and components of resistance to Cercospora zeae maydis derived from maize inbred VO613Y. Phytopathology 96:593-598.
Latterell F M, Rossi A E (1983). Gray leaf spot of corn: a disease on the move. Plant Dis 67:842-847.
Maroof M S, Yue Y G, Xiang Z X, Stromberg E L, and Rufener G K (1996) Identification of quantitative trait loci controlling resistance to gray leaf spot disease in maize. Theoretical and Applied Genetics, 93(4), 539-546.
Myles S, Peiffer J, Brown P J, Ersoz E S, Zhang Z, Costich D E, and Buckler E S (2009) Association mapping: critical considerations shift from genotyping to experimental design. The Plant Cell Online, 21(8), 2194-2202.
Nordborg M and Tavaré S (2002) Linkage disequilibrium: what history has to tell us. TRENDS in Genetics, 18(2), 83-90.
Pan Q, All F, Yang X, Li J, Yan J (2012) Exploring the genetic characteristics of two recombinant inbred line populations via high-density SNP markers in maize. DOI: 10.1371/journal.pone.005277.
Shi L Y, Li X H, Hao Z F, Xie C X, Ji H L, Lu X L, Zhang S H, Pan G T (2007) Comparative QTL mapping of resistance to gray leaf spot in maize based on bioinformatics. Agricultural Sciences in China. 6: 1411-1419.
Tehon L R, & Daniels E (1925) Notes on the parasitic fungi of Illinois: II. Mycologia, 17(6), 240-249.
Thompson D L, Bergquist R R, Payne G A, Bowman D T, and Goodman M M (1987) Inheritance of resistance to gray leaf spot in maize. Crop Science, 27(2), 243-246.
Ulrich, J F, Hawk, J A, and Carroll, R B (1990) Diallel analysis of maize inbreds for resistance to gray leaf spot. Crop Science, 30(6), 1198-1200.
Van Ooijen J W: MapQTL® 6 (2009) Software for the mapping of quantitative trait loci in experimental populations of diploid species. Kyazma B V, Wageningen, The Netherlands.
Wang L, Wang A, Huang X, Zhao Q, Dong G, et al. (2011) Mapping 49 quantitative trait loci at high resolution through sequencing-based genotyping of rice recombinant inbred lines. Theor Appl Genet 122: 327-340. doi: 10.1007/s00122-010-1449-8.
Wang S, Basten C J, and Zeng Z B (2011) Windows QTL Cartographer 2.5. Department of Statistics, N.C. State University, Raleigh, N.C.
Ward, J M J, Stromber E L, Nowell D C, Nutter F W (1999) Gray leaf spot: a disease of global importance in maize production. Plant Dis. 83:884-895.
Wu T T, Chen Y F, Hastie T, Sobel E, and Lange K (2009) Genome-wide association analysis by lasso penalized logistic regression. Bioinformatics, 25(6), 714-721.
Zwonitzer J C, Coles N D, Krakowsky M D, Arellano C, Holland J B, McMullen M D, Pratt R C, and Balint-Kurti P J (2010) Mapping resistance quantitative trait Loci for three foliar diseases in a maize recombinant inbred line population-evidence for multiple disease resistance?. Phytopathology, 100(1), 72-79.

We claim:

1. A method of identifying a maize plant that displays increased gray leaf spot resistance, the method comprising: a) detecting in germplasm of the maize plant at least one allele of a marker locus wherein the marker locus can be selected from marker loci within each chromosomal interval 1.1-10.2:
(1.1) comprising and flanked by PZE-101025686 and PZE-101026265;
(1.2) comprising and flanked by DAS-PZ-14748 and bz2-2;
(2.1) comprising and flanked by PZE-102013511 and DAS-PZ-32659;
(2.2) comprising and flanked by PZE-102040682 and Mo17-12859;
(2.3) comprising and flanked by PZE-102070420 and Mo17-13313;
(2.4) comprising and flanked by PZE-102072947 and PZE-102073407;
(2.5) comprising and flanked by PZE-102078235 and PZE-102079631;
(2.6) comprising and flanked by PZE-102088257 and PZE-102103382;
(3) comprising and flanked by PZE-103052576 and PZE-103057593;
(4.1) comprising and flanked by PZE-104093278 and DAS-PZ-8846;
(4.2) comprising and flanked by DSDS0099-1 and PZE-104105141;
(5) comprising and flanked by PZE-105166071 and DAS-PZ-14276;
(6.1) comprising and flanked by DAS-PZ-18055 and PZE-106101510;
(6.2) comprising and flanked by Mo17-12530 and Mo17-14401;
(7.1) comprising and flanked by PZE-107004762 and PZE-107004893;
(7.2) comprising and flanked by DAS-PZ-11250 and PHM4080.15;
(8.1) comprising and flanked by PZE-108006063 and PZE-108006412;
(8.2) comprising and flanked by PZE-108020151 and PZE-108020416;
(8.3) comprising and flanked by PZE-108022528 and PZE-108023337
(8.4-8.12) comprising and flanked by PZE-108047170 and PZE-108051324;
(9.1) comprising and flanked by PZE-109016836 and PZE-109017324;
(9.2) comprising and flanked by PZE-109083580 and PZE-109084648;
(10.1) comprising and flanked by PZE-110000036 and PZE-110000803; and
(10.2) comprising and flanked by PZE-110000803 and PZE-110001270; and,
b) the at least one allele within each chromosomal interval is associated with increased gray leaf spot resistance.

2. The method of claim 1, wherein at least one marker locus is selected from each of the groups 1.1-10.2 consisting of:
(1.1) chr1_{—}15269379;
(1.2) PZE-101188909;
(2.1) chr2_{—}6858691;
(2.2) PZE-102041193;
(2.3) PZE-102072013;
(2.4) chr2_{—}44697986;
(2.5) PZE-102079279;
(2.6) PZE-102088902;
(3) PZE-103053562;
(4.1) PZE-104093278;
(4.2) Chr4_{—}180264145;
(5) PZE-105165816;
(6.1) PZE-106100504;
(6.2) PZE-106107639;
(7.1) PZE-107004786;
(7.2) PZE-107020739;
(8.1) chr8_{—}7675588;
(8.2) PZE-108020413;
(8.3) PZE-108022834;
(8.4) PZE-108047366;
(8.5) GLS_chr8_{—}80296742;
(8.6) GLS_chr8_{—}80499765;
(8.7) PZE-108048175;
(8.8) PZE-108048978;
(8.9) GLS_chr8_{—}83335579;
(8.10) GLS_chr8_{—}86463733;
(8.11) GLS_chr8_{—}87640198;
(8.12) PZE-108050255;
(9.1) PZE-109017122;
(9.2) PZE-109084575;
(10.1) PZE-110000028; and,
(10.2) PZE-110000899.

3. A maize plant identified by the method of claim 1.

4. A method of identifying a maize plant that displays increased gray leaf spot resistance, the method comprising: a) detecting in germplasm of the maize plant a haplotype comprising alleles at one or more marker loci, wherein the marker locus can be selected from marker loci within each chromosomal interval 1.1-10.2:
(1.1) comprising and flanked by PZE-101025686 and PZE-101026265;
(1.2) comprising and flanked by DAS-PZ-14748 and bz2-2;
(2.1) comprising and flanked by PZE-102013511 and DAS-PZ-32659;
(2.2) comprising and flanked by PZE-102040682 and Mo17-12859;
(2.3) comprising and flanked by PZE-102070420 and Mo17-13313;
(2.4) comprising and flanked by PZE-102072947 and PZE-102073407;
(2.5) comprising and flanked by PZE-102078235 and PZE-102079631;
(2.6) comprising and flanked by PZE-102088257 and PZE-102103382;
(3) comprising and flanked by PZE-103052576 and PZE-103057593;
(4.1) comprising and flanked by PZE-104093278 and DAS-PZ-8846;
(4.2) comprising and flanked by DSDS0099-1 and PZE-104105141;
(5) comprising and flanked by PZE-105166071 and DAS-PZ-14276;
(6.1) comprising and flanked by DAS-PZ-18055 and PZE-106101510;
(6.2) comprising and flanked by Mo17-12530 and Mo17-14401;
(7.1) comprising and flanked by PZE-107004762 and PZE-107004893;
(7.2) comprising and flanked by DAS-PZ-11250 and PHM4080.15;
(8.1) comprising and flanked by PZE-108006063 and PZE-108006412;
(8.2) comprising and flanked by PZE-108020151 and PZE-108020416;
(8.3) comprising and flanked by PZE-108022528 and PZE-108023337
(8.4-8.12) comprising and flanked by PZE-108047170 and PZE-108051324;
(9.1) comprising and flanked by PZE-109016836 and PZE-109017324;
(9.2) comprising and flanked by PZE-109083580 and PZE-109084648;
(10.1) comprising and flanked by PZE-110000036 and PZE-110000803; and
(10.2) comprising and flanked by PZE-110000803 and PZE-110001270; and,
b) the haplotype is associated with increased gray leaf spot resistance.

5. The method of claim 4, wherein at least one marker locus is selected from each of the groups 1.1-10.2 consisting of:
(1.1) chr1_{—}15269379;
(1.2) PZE-101188909;
(2.1) chr2_{—}6858691;
(2.2) PZE-102041193;
(2.3) PZE-102072013;
(2.4) chr2_{—}44697986;
(2.5) PZE-102079279;
(2.6) PZE-102088902;
(3) PZE-103053562;
(4.1) PZE-104093278;
(4.2) Chr4_{—}180264145;
(5) PZE-105165816;
(6.1) PZE-106100504;
(6.2) PZE-106107639;
(7.1) PZE-107004786;
(7.2) PZE-107020739;
(8.1) chr8_{—}7675588;
(8.2) PZE-108020413;
(8.3) PZE-108022834;
(8.4) PZE-108047366;
(8.5) GLS_chr8_{—}80296742;
(8.6) GLS_chr8_{—}80499765;
(8.7) PZE-108048175;
(8.8) PZE-108048978;
(8.9) GLS_chr8_{—}83335579;
(8.10) GLS_chr8_{—}86463733;
(8.11) GLS_chr8_{—}87640198;
(8.12) PZE-108050255;
(9.1) PZE-109017122;
(9.2) PZE-109084575;
(10.1) PZE-110000028; and,
(10.2) PZE-110000899.

6. A maize plant identified by the method of claim 4, wherein the maize plant comprises within its germplasm a haplotype associated with increased gray leaf spot resistance wherein the haplotype comprises alleles at one or more marker loci located within each chromosomal interval 1.1-10.2:
(1.1) comprising and flanked by PZE-101025686 and PZE-101026265;
(1.2) comprising and flanked by DAS-PZ-14748 and bz2-2;
(2.1) comprising and flanked by PZE-102013511 and DAS-PZ-32659;
(2.2) comprising and flanked by PZE-102040682 and Mo17-12859;
(2.3) comprising and flanked by PZE-102070420 and Mo17-13313;
(2.4) comprising and flanked by PZE-102072947 and PZE-102073407;
(2.5) comprising and flanked by PZE-102078235 and PZE-102079631;
(2.6) comprising and flanked by PZE-102088257 and PZE-102103382;
(3) comprising and flanked by PZE-103052576 and PZE-103057593;
(4.1) comprising and flanked by PZE-104093278 and DAS-PZ-8846;
(4.2) comprising and flanked by DSDS0099-1 and PZE-104105141;
(5) comprising and flanked by PZE-105166071 and DAS-PZ-14276;
(6.1) comprising and flanked by DAS-PZ-18055 and PZE-106101510;
(6.2) comprising and flanked by Mo17-12530 and Mo17-14401;
(7.1) comprising and flanked by PZE-107004762 and PZE-107004893;
(7.2) comprising and flanked by DAS-PZ-11250 and PHM4080.15;
(8.1) comprising and flanked by PZE-108006063 and PZE-108006412;
(8.2) comprising and flanked by PZE-108020151 and PZE-108020416;
(8.3) comprising and flanked by PZE-108022528 and PZE-108023337
(8.4-8.12) comprising and flanked by PZE-108047170 and PZE-108051324;
(9.1) comprising and flanked by PZE-109016836 and PZE-109017324;
(9.2) comprising and flanked by PZE-109083580 and PZE-109084648;
(10.1) comprising and flanked by PZE-110000036 and PZE-110000803; and
(10.2) comprising and flanked by PZE-110000803 and PZE-110001270.

7. A method of marker assisted selection comprising:
a. obtaining a first maize plant having at least one allele of a marker locus, wherein the marker locus is located within each chromosomal interval 1.1-10.2:
(1.1) comprising and flanked by PZE-101025686 and PZE-101026265;
(1.2) comprising and flanked by DAS-PZ-14748 and bz2-2;
(2.1) comprising and flanked by PZE-102013511 and DAS-PZ-32659;
(2.2) comprising and flanked by PZE-102040682 and Mo17-12859;
(2.3) comprising and flanked by PZE-102070420 and Mo17-13313;
(2.4) comprising and flanked by PZE-102072947 and PZE-102073407;
(2.5) comprising and flanked by PZE-102078235 and PZE-102079631;
(2.6) comprising and flanked by PZE-102088257 and PZE-102103382;
(3) comprising and flanked by PZE-103052576 and PZE-103057593;
(4.1) comprising and flanked by PZE-104093278 and DAS-PZ-8846;
(4.2) comprising and flanked by DSDS0099-1 and PZE-104105141;
(5) comprising and flanked by PZE-105166071 and DAS-PZ-14276;
(6.1) comprising and flanked by DAS-PZ-18055 and PZE-106101510;
(6.2) comprising and flanked by Mo17-12530 and Mo17-14401;
(7.1) comprising and flanked by PZE-107004762 and PZE-107004893;
(7.2) comprising and flanked by DAS-PZ-11250 and PHM4080.15;
(8.1) comprising and flanked by PZE-108006063 and PZE-108006412;
(8.2) comprising and flanked by PZE-108020151 and PZE-108020416;
(8.3) comprising and flanked by PZE-108022528 and PZE-108023337
(8.4-8.12) comprising and flanked by PZE-108047170 and PZE-108051324;
(9.1) comprising and flanked by PZE-109016836 and PZE-109017324;
(9.2) comprising and flanked by PZE-109083580 and PZE-109084648;
(10.1) comprising and flanked by PZE-110000036 and PZE-110000803; and
(10.2) comprising and flanked by PZE-110000803 and PZE-110001270; and the allele of the marker locus is associated with increased gray leaf spot resistance;
b. crossing the first maize plant to a second maize plant;
c. evaluating the progeny for the at least one allele; and
d. selecting progeny plants that possess the at least one allele.

8. The method of claim 7, wherein at least one marker locus is selected from each of the groups 1.1-10.2 consisting of:
(1.1) chr1_{—}15269379;
(1.2) PZE-101188909;
(2.1) chr2_{—}6858691;
(2.2) PZE-102041193;
(2.3) PZE-102072013;
(2.4) chr2_{—}44697986;
(2.5) PZE-102079279;
(2.6) PZE-102088902;
(3) PZE-103053562;
(4.1) PZE-104093278;
(4.2) Chr4_{—}180264145;
(5) PZE-105165816;
(6.1) PZE-106100504;
(6.2) PZE-106107639;
(7.1) PZE-107004786;
(7.2) PZE-107020739;
(8.1) chr8_{—}7675588;
(8.2) PZE-108020413;
(8.3) PZE-108022834;
(8.4) PZE-108047366;
(8.5) GLS_chr8_{—}80296742;
(8.6) GLS_chr8_{—}80499765;
(8.7) PZE-108048175;
(8.8) PZE-108048978;
(8.9) GLS_chr8_{—}83335579;
(8.10) GLS_chr8_{—}86463733;
(8.11) GLS_chr8_{—}87640198;
(8.12) PZE-108050255;
(9.1) PZE-109017122;
(9.2) PZE-109084575;
(10.1) PZE-110000028; and,
(10.2) PZE-110000899.

9. A maize progeny plant selected by the method of claim 7 wherein the plant has at least one allele of a marker locus wherein the marker locus is located within each chromosomal interval 1.1-10.2:
(1.1) comprising and flanked by PZE-101025686 and PZE-101026265;
(1.2) comprising and flanked by DAS-PZ-14748 and bz2-2;
(2.1) comprising and flanked by PZE-102013511 and DAS-PZ-32659;
(2.2) comprising and flanked by PZE-102040682 and Mo17-12859;
(2.3) comprising and flanked by PZE-102070420 and Mo17-13313;
(2.4) comprising and flanked by PZE-102072947 and PZE-102073407;
(2.5) comprising and flanked by PZE-102078235 and PZE-102079631;
(2.6) comprising and flanked by PZE-102088257 and PZE-102103382;
(3) comprising and flanked by PZE-103052576 and PZE-103057593;
(4.1) comprising and flanked by PZE-104093278 and DAS-PZ-8846;
(4.2) comprising and flanked by DSDS0099-1 and PZE-104105141;
(5) comprising and flanked by PZE-105166071 and DAS-PZ-14276;
(6.1) comprising and flanked by DAS-PZ-18055 and PZE-106101510;
(6.2) comprising and flanked by Mo17-12530 and Mo17-14401;
(7.1) comprising and flanked by PZE-107004762 and PZE-107004893;
(7.2) comprising and flanked by DAS-PZ-11250 and PHM4080.15;
(8.1) comprising and flanked by PZE-108006063 and PZE-108006412;
(8.2) comprising and flanked by PZE-108020151 and PZE-108020416;
(8.3) comprising and flanked by PZE-108022528 and PZE-108023337
(8.4-8.12) comprising and flanked by PZE-108047170 and PZE-108051324;
(9.1) comprising and flanked by PZE-109016836 and PZE-109017324;
(9.2) comprising and flanked by PZE-109083580 and PZE-109084648;
(10.1) comprising and flanked by PZE-110000036 and PZE-110000803; and
(10.2) comprising and flanked by PZE-110000803 and PZE-110001270.


SEQUENCE LISTING (78 sequences)

SEQ ID NO: 1 - DNA, 401 bp, Zea mays
acgccggctgccggccatgtacatttatgcagtcgtcggcgtcgaagcttctcaaggagacgtgcgcctacatcaagagcctgcaccgggaggtggacgacctctcggaacggctgtcggggctcatgtcgaccatggacaacgacagcccccaggccgagatcatccggagcctcctccggtgacccggccatgccgccmaggcgcgcgcgcgcggggctggccctcttcgattctgcctgccatctagtagctagctgcagcagccagcgcagctgcactgaagacgacgacgacaacgcgacgaccttaattatgttcctttgttcttcttccggtgtgtgtgagtgtgtgtgtttccgtgtttaattaatttgagagcgaggagctggatggagcct

SEQ ID NO: 2 - DNA, 101 bp, Zea mays
ttcagggtgctgatttatttttccgggcagtaatctcgaacatggaaaagrtttatttgagcaggaatcctacagccaaaaccattctggagcttgtacga

SEQ ID NO: 3 - DNA, 401 bp, Zea mays
tgccccatcgacgcgctgaagctcggcgcgtgcgtggacattctcgggaacgaggtgcacatcggcgacgccaacgtcaagtgctgcccgctcgtgaagggcatcgccgggctctccgcggcggcgtgcctgtgcaccgccatcaaggccaaggtgctggacatttccgtctacgtgcccatcgcgctggaggtgctcgtyaactgcggctgcgaggtcccgccaggctacaaatgcacttaaacgactccattccatttcatcccatctcaccatgcgtgcacagcgaggtctccttttgccctcccgaatgcaaagtttgaaaggctcgtcgacggatttgggtctccttttgcccacccgaatgcaaagtttcgaaggctcgtcgacggattgggctc

SEQ ID NO: 4 - DNA, 101 bp, Zea mays
gaagctgtcggcgtctccattgaagtccatgtccatgttcgtctgtcgatrgagccgtacgcccgtaccactgtacgcagcggaggaagaggatggcacaa

SEQ ID NO: 5 - DNA, 101 bp, Zea mays
tagaaatacttgatctgcatgcaatgcaacgcgacaacaaccgagtccggyccgtcagataatattgagatgccaggcccaggatgcgggggccatgcagt

SEQ ID NO: 6 - DNA, 401 bp, Zea mays
ctcccggtgagcggcgagatgttgcccatcttgaccatggacttggcgaagtggtcgaagaagaggccctggtcggcggcgtagcggtggacgaggtccatggtctcgcggctctgcgtgagcaggatctcgtcggagctgagcagcccgttcatggccaggatgttgtggtagtactggttgtcgaaccggaactgggtyaccaggtccagcgcgaacaggttctggtcgccgcccgaccgcgggcaccggccccgcagctccgccgcgtacgccgggttcagcgtccggtccacctgcccgttgttgttctggccgtacagccgctgccggaagctcacgcaccgcgagtcgccgatggtgtgcccccctgcgttcagttgttcgttgcagttggaagg

SEQ ID NO: 7 - DNA, 101 bp, Zea mays
gtgcgcgtgcgcgtgcgcctacgccgtccccgcccgcccttggttttcggyactttggattgtatggagggtacggctgggcagttcttcctcttcctcgt

SEQ ID NO: 8 - DNA, 101 bp, Zea mays
cttcgcgcggcaccgctggggctctgcgtggccgccatgaccgtcatgctmcgtaaccagcagagcaacgagtacggcgccgtcgcctactccgacctggg

SEQ ID NO: 9 - DNA, 101 bp, Zea mays
cgctgggcagcctctgcgacgagctgagtgcgcagatgaaagacgcgacgmttgtgtacacggacctgttccccatcaagtatggtcttgtcgccaaccac

SEQ ID NO: 10 - DNA, 101 bp, Zea mays
ccttcggcaacctccccgagatcacggcctcgctgcaggcccaggcccagrccaacaacaacaagtcctccgccgtcgccagcggcgacatccacgccgcc

SEQ ID NO: 11 - DNA, 401 bp, Zea mays
gtttgtgtcgtacaacgctacatttgccattcgagcactcctagggaaaaatagcgtcgttagactgtcagcaacgcactgccctacggctcccctcccctattactgtgctcatgcaaggggcaaaaatcacccccttcctcccgcatcgcctctccctgcagtaccccctacctggggttcctcttctctctcacgctrgatcaggcggagcactattcatcccccaacctttccttccggaggatggcagcgagcgcgccttggcgaagaaacctcccgctccgttgcggtgggtttggactccagcgcggaggcctggacacgacaatgtgcaaggcggaggaaggccgactcggatccgctcgatctcgccgacgacgaggcccaacaacaggacg

SEQ ID NO: 12 - DNA, 101 bp, Zea mays
tggacgcatgcagatgcagacgcatgatgcatgccgacctgattggacgcwgccatgtcgtcggcgacgcccttcttgccgctaccaaccttcttggcggg

SEQ ID NO: 13 - DNA, 101 bp, Zea mays
aaatgatgtgtcctacatcttacagaaggcaatcccactctgcagatgccygcaaagttggagcctccaaacaaatcactacagatagtgtacatagtgtg

SEQ ID NO: 14 - DNA, 101 bp, Zea mays
agaagaagtgtatcaaaagtcaagaatatgcagcagagcatggtttgcctrttctgaaaaacgtactccttcccaagacaaagggtttcaattgctgtttg

SEQ ID NO: 15 - DNA, 101 bp, Zea mays
ttgcagttgagcacgagttttgctttgccttgtgtcctgtactgccgcgcrtgcggcccctgctgaaataatcacagtgccgttttttacaagacaaagga

SEQ ID NO: 16 - DNA, 101 bp, Zea mays
gaggaatcgcttcgacggtcgacggtcaggcgtcctgttccgctgcggcgraaggaggcgcggatcacctagggctacggaggccgctcgatgaggggcga

SEQ ID NO: 17 - DNA, 401 bp, Zea mays
ttggagagccaagggcggcgcggcagcagcgggtttctcggtaaatctcggttcttgatgagttatttagttgaggttttgatattgggaagcgctagattaatgtttccatgagtaattcctttcgcacggttgcatttgttgatgtttaggtcggactagattgtttccatcgagatggtgggcggcggagggcattcrtcgatggaccaccctggcgccaaggacctcgagcgcggcgagctgcgccgtggcgcgcctgagtttgcggacggcgacgatggagacggggaagaaagccagtacttctcggacgcggaggaccggtcgtggccgtcgcactcgcgccacgactccaccgcctacgaggactacgtctcgctgtgcgtgtccgcccgcgc

SEQ ID NO: 18 - DNA, 101 bp, Zea mays
tgaattgcaaagctgcaaacggcacatcacccagaccacaccgatttctcrcagctttaccagccttgctgtcagcgaagaccatggaacatgttaataat

SEQ ID NO: 19 - DNA, 101 bp, Zea mays
cgccgatggatggatagacagcaaattccggtgagcacatcgatccgtttyattccatgcgccgatcgatgcatataggtgcatgaaaacttaattactca

SEQ ID NO: 20 - DNA, 101 bp, Zea mays
ccattgcaaatcctagcagcatggtcttctttttctttcccttgcgttttrttatgcgcccttaacagctcggtgtaggtttagaaggcacaacaaaactg

SEQ ID NO: 21 - DNA, 501 bp, Zea mays
agggagagccatcacagctggctcccattgatgtcttcgtcagtacagtggatccattgaaggaacctccactgatcacagccaacactgttttgtccattctttctgtggattaccctgttgacaaagtgtcatgctatgtttctgatgatggttcagctatgctgacttttgagtctctctcagaaaccgcagaatttgctagaaagtgggttcccttttgtaagaagcacaatattgaaccaagagcyccagaattttactttgctcaaaaaatagattacctgaaggacaaaattcaaccttcatttgttaaggaaagacgcgcaatgaaggtaaactgctatctcatggttacatcaccattgctctaccttcctctcttttttaactctggatatgcattttttcagagggagtatgaagaattcaaagtaagaatcaatgcccttgttgccaaagcacagaaagtgcctgaagaggggtggaccatggctgatg

SEQ ID NO: 22 - DNA, 501 bp, Zea mays
ttatttgcaaaaaggtagttataaattctaaaaaataaatgcatagccaagggttgatttccacattatgttgaacatgtgcctaacgtaaacacattgcaaaaataacagatgtggatgagctatttacctcgattgccttgcttggggctacccggatgacattaacaacgttaccacggaacaacccagtccatccctcatgcttcatgatagactggaacacctccgtcgtcgaattcccattactkcccaccatcaaatgcgtcctgatcgtctccaaaggtgcaacagcagttcttgacactgcgcctgcgattgctccactgatcagtctcttgaggtgatgattcccaaccttaatcttgagcttcacgactttcttcttccccttgtccctcaacccccccacaccttcccccggcagaggtgtctcaacaacctccggcgagacgtacttcatgtatgggtcccgtggcgtctccggcgactgggacgaag

SEQ ID NO: 23 - DNA, 101 bp, Zea mays
taatcgccaactgtttttcatatgttgcaggtgaaagaaagtgaggcagcycctttgatctcagactctttgagcaaagttgaaaatgggggcggtgttgt

SEQ ID NO: 24 - DNA, 101 bp, Zea mays
tggcctctctggacgtgttccgcggcttcaccgttgccgtgagtggaaacycgatcatgctagttttttgctcggatgctccaaaattcaggcgtggactc

SEQ ID NO: 25 - DNA, 501 bp, Zea mays
tgaggagtcactcaatccaaaatgatgtggtgtataatgagtccattcctcaaatttgctgggatgaccttatttctcatattagtactaactaactataaggaatgactccattccttatttcattccacaaatcaaataaaaaaagagaagtgagaagacgatggactagctcattcctcaaaccaaacactttataattaattcattctccataaccagtccccaatttaatttaactactatccttrccgcactcttaactatacgaggataaagtagaaatatacctttcgaagcatctcttctgcagatggcggttcctcacgctgcaggctgatgaaataagactggagcttcttggcagcctccatgaaatcccttgcgtgcctctcaacatcaactgcccaatttagtagagactacaacacttagataccctgtttggcaggaacgaatgagaaaatgcaattaaatttctctaataagcattgcacacta

SEQ ID NO: 26 - DNA, 501 bp, Zea mays
tattccaatttgatcacaagttagaccctctcttaatgttaattacaaatttatccaatagatgaaagaagcaaccccacatattgtttaacgttatatatctttgaacatcaaaactcaccaatgtctgaggtataagcgacatggtaagatatcatctctccatattttgtcaatattgtaccgtccatagtacttgttatatatttccttgcttcctgtagaagtagccactcaaagtttcaataagyaattgactatgaaattaaatatgatttggtattgtctcaagcaattctctagaagttagcaacctatatctatattgccttaaaagaacaacttccatctaaagaaatatgttttgattcgccagaagaaaaaaaagacagaaaaatgttcagcataaagctttgttagtcatcataaatttatttgagagggattcaaacatatctagcaactacaatctagtactagtatttcgttagacaggggggg

SEQ ID NO: 27 - DNA, 501 bp, Zea mays
gggctcagcaaatcatcaagtggaagaccagggtccaatctttgagcaattttaattaatttttctgcgcgctgcctatccccagatgcaaatgcagattttgcaagcttgacagatctcaatgcctcatctttgttcccatccatgagtccaatcagctctggtgaaataggttgaaacctcaaactgatgacctgtgaaacaagaatactggattaacaaaacaaagcaaactatcatgtaataaaacyggctaacaaacaccaatttccatgagatgaagtgaagtgatatctaaatctcagcttagaaatctagtagtcataaacaagttgtaggcaaagagtacaaaaacatcacaggaatttcacagaaatcggttcatttcacaggaaaaaagcaaggaacacgaaaatttctggcgttccaaaggggggctaaatattagacggatcctctatagccacatgagttatgcatgtcataaacaaaacagaacta

SEQ ID NO: 28 - DNA, 101 bp, Zea mays
tctagcgcagccgcggcggcgggagtgggtgcgatggcattgatcgtgcakggggaggacacggccttcgggtctctggagtggtgggcgtacgcgggcat

SEQ ID NO: 29 - DNA, 101 bp, Zea mays
caatgcgcatcagtatgacagtagtaggatccacggacatttgccacacakctgagggaactgcaaagttttcacctggtgaattgttgggaaggacgacg

SEQ ID NO: 30 - DNA, 101 bp, Zea mays
aacgccttcgacgggcctctcagggaggccgtggccgcgctcagcaagcgycgcacctacctgctcgagatgaacggcttctagtctccttggccaatggc

SEQ ID NO: 31 - DNA, 101 bp, Zea mays
gacttgaacctcccaggcgacgatgaggaagcccatgatgatggtgatgawgacaagagcagtggcagccacgaaaactatctagagggatctgtcggaaa

SEQ ID NO: 32 - DNA, 101 bp, Zea mays
catgctctccagctgcttctgcgcagcgcgggatagaggacaaaaggcagrcaggtgttaattagagtacgagcgagcaagcagcataaaatgcagcggct

SEQ ID NO: 33 - DNA, 101 bp, Zea mays
cacgcacctgcgtcgccccaatcgagagaccgtatggtcgcggcacgccaygccgtccacgccgcgtcaacacgtttgaccggcccagtcctcgtttacgg

SEQ ID NO: 34 - DNA, 201 bp, Zea mays
agacatgcttgtccattgggctgaactgtctggttcaggcagatctccatctatcccaggccgacgagaaagtcggcggtggcacagccgcacaggcaacraccaacatagcatacttgaaaatggctggcgtgtgcatagcatacttgaaatcttaatgaacttgtttgtcagtcatggatgctggattacgtgtggttg

SEQ ID NO: 35 - DNA, 101 bp, Zea mays
gccgggcaggggcaaatgccggcgcgcgacggcagcgggtacggtcggctrgattacaagtggaaggttcgctcgtctcgtggtgggagtttgaaaacacg

SEQ ID NO: 36 - DNA, 101 bp, Zea mays
gaaattaatctattttgcaccacgctgggcagcatatatatatagtatgcycaggaaccatgcatatacagatcaagataaaaggcataccacacccggtc

SEQ ID NO: 37 - DNA, 101 bp, Zea mays
accacgtttcgtgcgcgcttctgagctccctcggcccgtggtacagtcttratcacacattcctcaaacactgctgctgcattcccaaatataaaatccac

SEQ ID NO: 38 - DNA, 101 bp, Zea mays
agtagagctcgttcatgtaatgtgagacctcacacgatgtgtaatgcctakggatccggaacaatatatgctactcattcttgagatcgcgtgtaggtaaa

SEQ ID NO: 39 - DNA, 101 bp, Zea mays
gccaaccctgccggacagcgccccgttgcggtcacacacctctgccttttyggtgtgattatccgtaccccgccacggtcccttttgaggtgcactggcag

SEQ ID NO: 40 - DNA, 101 bp, Zea mays
gtcattggtttgaacttggacgagagcaaagtacgtcgtgctggacctgcygagaatgttcgtgtcaaattgtccggagttgaggaggaggatgtaatgtc

SEQ ID NO: 41 - DNA, 101 bp, Zea mays
tggtcaaacttgaaccagcagcaaaattggccgccgggacttgatgcacgrgtgaacattgttcgcctcaataattgagcaggaaagtggaaattacgcat

SEQ ID NO: 42 - DNA, 97 bp, Zea mays
tagacacctgcttcagaaggtccagcggggcttcctcgcctgatgaatttmccttccttaaatatatacagcataggaatgccagtagacaactcaa

SEQ ID NO: 43 - DNA, 101 bp, Zea mays
gggttgtgtgtctgtgtgattgtgatagaatccaaagacgcaagcggctgmaggcagcagcgccgcgcaggcgttgtggccttgtgggagaggaaaaagag

SEQ ID NO: 44 - DNA, 201 bp, Zea mays
agtgtttgaatatctcaactaattttagccactaactattagttttattgcattcaaacaccactaggtccaaccgtatatatcaccacctgtatgggtaytttattcctgtcttttacagacttgtgtacaacaatttttttccctgtatatatcacctgcctaccccgccggttctgttttaatttgaatctggatttt

SEQ ID NO: 45 - DNA, 121 bp, Zea mays
agcggagggagtacggtcatggctctgtctctcatgcgacatgcgagtaatgaatactacwaataaatgcatgttgtttgatatatatactcctcgctgaggagaagagaatccggcctgg

SEQ ID NO: 46 - DNA, 101 bp, Zea mays
gagaatcgcaggtgcagggccccggccccggccccgtcagcgtcagagtgycactggcgagtgttgacgcgacgacgagcctcgccgcctatatattgcgg

SEQ ID NO: 47 - DNA, 201 bp, Zea mays
gaccccgcttcccctcaaatattttatatacatatttttattttattttatttactgtcaatattttatttattattttccgcgaactctctgtcagcttstaataaataatcacccttgttgtcgtgcaggaaaggcgtactcttcaatttaatttttgagtatcaggccataatagtttatccggttccttgaactcgt

SEQ ID NO: 48 - DNA, 101 bp, Zea mays
aagtcgcatggccatgggatcaccgaccggaagagaagctagagcaaacaragagagagagagaacccaagcctcgatactatgtacaagataaagataac

SEQ ID NO: 49 - DNA, 101 bp, Zea mays
catgagcggtccatgatggatatagctagattgaatattaatgagcaccarattaagggacaccaatcagccgccgtacgcacagtaggagtgccaaataa

SEQ ID NO: 50 - DNA, 101 bp, Zea mays
gtggtggacctgcaggacgtgttcgtgcgcctcaccttcgatctcaccgcyatgttcgtgttcggcatcgaccccggctgcctcgcccctgacttccccta

SEQ ID NO: 51 - DNA, 101 bp, Zea mays
aattgtaaaggaaaaagtttgtagcctattgcagcagcatacatgctaacygagtaatatgaaaagcagtatgaagctagtactctttcaattcaaattat

SEQ ID NO: 52 - DNA, 101 bp, Zea mays
ccagccgactccacagttcatccggtgttccacgtctcccagctcaagaargttgtctcttctaagcacccggtgagtaccacactacctgacgacacagc

SEQ ID NO: 53 - DNA, 101 bp, Zea mays
gtcaagagcctgatccaggatatctgaagtccttgatgctgctttagtgcrcatccgtccgtgtgttccattcggaggcggtcaaatgctacattcgccta

SEQ ID NO: 54 - DNA, 101 bp, Zea mays
gtgcttaaaacacactttctcaacttgaaaacagagttttgaggatcttcrtactataggcctccaacaatcgttagtttttcacttttcaaataataacg

SEQ ID NO: 55 - DNA, 101 bp, Zea mays
atgtcatactgtattctcggagaaccgttggaatctgcagattgttaagtycaatcaggatttctcttcattctgatagttcggtagtcaacgaaacaaaa

SEQ ID NO: 56 - DNA, 101 bp, Zea mays
cacacaacacacacacacagacagagagggagagactgttacggctccgartgtgagaggtgcttacggctatcaagtgcggcgagggagtgagtaccttg

SEQ ID NO: 57 - DNA, 121 bp, Zea mays
tccccgtrctgctcctccccgacggccgcgccatatgcgagtccgcagtcatcrtccagtrcatcgaggacgtggcgcgtgraagcggcggcgcmgaggctrgcagcctgctgctsccgga

SEQ ID NO: 58 - DNA, 201 bp, Zea mays
ggccgcagaatcgccgcaggaaggagacaagccgtttggcagcgtaagtgttcgaggccaccgcttgattatttctccgtgctgtaacccgtagatggatrtgcaaccgttgcggtgtctgtacgtgcaggaggaggtgggcactcaggtgttggcggcgacgaggacgccgccgtggccgccgtgccctcctgaagaagg

SEQ ID NO: 59 - DNA, 121 bp, Zea mays
ttcaagcaaatgacgtgtgctttctctgcccgccggatgacaagtttgaacaggtgccatsagcacttttttccccttctttttcacttttgcgaatcaggatttcatccattcatcaggg

SEQ ID NO: 60 - DNA, 121 bp, Zea mays
caggattcgacggagccgaaggaaccagccgacaccgtgccgtacgccttaactgcacgtmctacgtggaagctaacgtttgcgtgcgtggctcgccgcgcgccgccggcccgcccccacg

SEQ ID NO: 61 - DNA, 101 bp, Zea mays
aaagcaatgggcaaagtgacgcggcgcttctataatgctcgcatctgcccycatcagcttggctgtgtcacctaaaagccttgtgctaaaataatctctag

SEQ ID NO: 62 - DNA, 101 bp, Zea mays
cgctacgccctcgaggaccatgtactcactgatgcgaacgcgccgacgcakgcctagtgccggatgaacagtgtggttctctcctggatcctcggcaccct

SEQ ID NO: 63 - DNA, 101 bp, Zea mays
ggatgagcgaccgaccaacctgtccgcgcatctccggtgatgtagtccgaygaagtgttggaataagaccacccaaccccagccggcgattccacgaacag

SEQ ID NO: 64 - DNA, 101 bp, Zea mays
gagcaatcgtcgcagcccaggatgaactttctccatactgactccatgcayagcttccccatgccacgaggaatctccatggccgccccacgagagtagag

SEQ ID NO: 65 - DNA, 201 bp, Zea mays
acctcctgttctgaatcagaggtaggaagcttttcgtggtggccgccccatattctgactctctccaacgccagcgacgcctgcaaaatagaaggtcaggkcaggccaatgaaagcgtgtttctggtgatagagatcgatcgtcgtcaccacctacctctgcttcccagtcggtcgtccagactatgtagctgaggatggc

SEQ ID NO: 66 - DNA, 101 bp, Zea mays
cctgcttgggtaggggagaggaaggggataggggtggagccgtgcagattrcagattagacgagttacagaaaagttgtagctggggaaaaaatatgagtt

SEQ ID NO: 67 - DNA, 201 bp, Zea mays
tccgagtcccatcggctgctccctctgccatactgatttctggaagcagcttctcgttttgagcacacgagaggatctggttcagaatgtgtgggaacggrccgtttctctgcaagatacctttggtttcatatatcctgatctgaattcccacccttttttccgctttggccaggtaggacctgaagcatcttgtaaccg

SEQ ID NO: 68 - DNA, 101 bp, Zea mays
caatatatgtgtgcatctatcgagtgcatgcatacaaggcaagctacgtamagcaagtcaatttggactcgaacatacatgtgcgattatttgggagccaa

SEQ ID NO: 69 - DNA, 121 bp, Zea mays
gactgtggagaccacgcgtcccgtcccgtcccagtcactgacaagttgattacggtcatgstagcccaacctgtcagtcacacaagaaaatgctcccggggcctcgtagcatctagcactc

SEQ ID NO: 70 - DNA, 101 bp, Zea mays
acgagtccccaagcagcagcagatgatggtcctaaaagatttcaacgggtrtctgcaccggcctgtcagagcacaaatcgaagcctagccgccactctcac

SEQ ID NO: 71 - DNA, 121 bp, Zea mays
agcagaagcagcccgtcatggtggcctgtatgaatcagcaggggtctcctatgtcctaggygttccatgcaccatcgaataaatccacccctcttcatgttagaatgggaaacggggatgt

SEQ ID NO: 72 - DNA, 101 bp, Zea mays
gcagcagcaagacgcaaacaggcaagggctaggccgcggcctgcaggggcrctgcggccgcttgggagctgggggctgggagcctgctggtcagtggacgc

SEQ ID NO: 73 - DNA, 101 bp, Zea mays
tgatagaaaaaaaaaactcattcaattgcagccattacgtgtgatagataycgatataacattgaaatgtaggtgggtgaagaatacttacaacattgacc

SEQ ID NO: 74 - DNA, 101 bp, Zea mays
ttcgtgtaaagagcaacgtacctctgcagcagctcgtttctagcatctacragacagttaagaaactcgttaacctgaaaaacaaggcagagagcatgtac

SEQ ID NO: 75 - DNA, 101 bp, Zea mays
ggccacggtgtacccgtggacccggtccatgtgactcacgtcctcgtcgayacagtccggcacctttccctcgcccttcctaccgttacccatcgggcatc

SEQ ID NO: 76 - DNA, 101 bp, Zea mays
cgatgaacaggcgcccagaggatgtcgacctgctccgggttattgtagttkccctggaccgtgtggtaagcctcgtgactttggtcgacccacgacgtccc

SEQ ID NO: 77 - DNA, 101 bp, Zea mays
tagaatgaaccgttgaacaccaccaggtggtattgggctgcatgcacgcayagataagctcacccacggttttcgacgaccacttccctcacgtcaaggtt

SEQ ID NO: 78 - DNA, 101 bp, Zea mays
gacaaacaagagattgctgcataaagccaaatcaacatgcttttccccagmaaatcagcgcatgaagaatcttcattcaatttctctgccaggggttttat